(19) United States
(12) Patent Application Publication
(10) Pub. No.: US 2008/0249043 A1
(43) Pub. Date: Oct. 9, 2008

(54) Methods and Compositions Involving Protein Kinase C-Delta

(76) Inventors: Ruey-Min Lee, Richmond, VA (US); Jihua Liu, Salt Lake City, UT (US); David Durrant, Richmond, VA (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/658,191

(22) PCT Filed: Jul. 22, 2005
(86) PCT No.: PCT/US05/26045

§ 371 Date: Jan. 31, 2008

Related U.S. Application Data
(60) Provisional application No. 60/590,808, filed on Jul. 23, 2004.

(51) Int. Cl. A61K 31/70; C12Q 1/48; A61P 43/00; A61K 31/69

(52) U.S. Cl. 514/44; 435/15; 514/64

(57) ABSTRACT

Disclosed are methods and compositions relating to the use of PKC-δ activators in the treatment of disease.

### CROSS REFERENCE TO RELATED APPLICATIONS

This application claims the benefit of priority of U.S. Provisional Application No. 60/590,808, filed Jul. 23, 2004, which application is incorporated herein by this reference in its entirety.

This invention was made with government support under federal grants NCI K08 CA79509, awarded by the NIH. The Government has certain rights to this invention.

### BACKGROUND OF THE INVENTION

In the post genomic era, the trend for developing novel cancer therapy is based on identification of molecular targets. In addition to utilizing kinases or other signal transduction mediators as targets, members of apoptotic pathways also serve as useful targets due to their ability to directly inducing cell death and minimize the development of drug resistance.

Apoptosis is a tightly regulated form of cell death, also called programmed cell death. Morphologically, it is characterized by chromatin condensation and cell shrinkage in the early stage. Then the nucleus and cytoplasm fragment, forming membrane-bound apoptotic bodies which can be engulfed by phagocytes.

Apoptosis is an important process during normal development. It is also involved in aging and various diseases such as cancer, AIDS, Alzheimer's disease and Parkinson's disease. During apoptosis, the cell is killed by a class of proteases called caspases. More than 10 caspases have been identified. Some of them (e.g., caspase 8 and 10) are involved in the initiation of apoptosis, others (caspase 3, 6, and 7) execute the death order by destroying essential proteins in the cell. The apoptotic process can be summarized as follows: activation of initiating caspases by specific signals; activation of executing caspases by the initiating caspases which can cleave inactive caspases at specific sites; and degradation of essential cellular proteins by the executing caspases with their protease activity.

Bortezomib (Velcade®), a targeted cancer drug, has been shown to be more effective than standard therapy at delaying disease progression in patients with cancer, for example multiple myeloma that has relapsed or become resistant to other treatments. Bortezomib works by blocking proteasomes, clusters of proteins necessary for cancer cell growth. What is needed in the art is a combination treatment therapy involving bortezomib to treat cancer.

### BRIEF SUMMARY OF THE INVENTION

This invention relates to compositions and methods that in one respect relate to methods for identifying molecules of interest that activate PKC-δ. Also disclosed are compositions and methods that in one respect relate to molecules having anti-cancer properties.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows inhibition of bortezomib-induced apoptosis by PKC-δ inhibitor rottlerin. U937 cells were treated with bortezomib at 0, 5, 10, and 50 ng/ml with and without PKC-δ inhibitor rottlerin (10 μM) or PKC-δ inhibitor Go6976 (200 nM) for 16 hours. Cells were harvested and fixed for TUNEL assays. The percentages of the TUNEL positive cells are indicated.

FIG. 2 shows bortezomib-induced changes of PKC-δ. (a) shows up-regulation of PKC-δ by bortezomib at early time. U937 cells were treated with PMA (200 nM) or bortezomib (100 ng/ml) for 2 and 4 hours and cells were harvest for western blotting with antibody against PKC-δ. Same blot was probed with actin antibody as loading controls. (b) shows bortezomib induces translocation of active PKC-δ fragment to mitochondria. U937 cells were treated with various concentrations of bortezomib and cells were subjected to subcellular fractionation to isolate mitochondria and cytoplasm. The whole cell lysates and fractions were analyzed with antibody against PKC-δ. (c) shows blocking caspases prevents the generation of active PKC-δ fragment. U937 cells were treated with bortezomib (50 ng/ml) and/or caspase inhibitor BAF (30 μM) for 16 hours. Whole cell lysates were analyzed by western blotting with the PKC-δ antibody. (d) shows induction of PKC-δ translocation to mitochondria. Cells were treated with PMA (200 nM) for 6, 9 and 12 hours and subjected to subcellular fractionation. Western blot was probed with the PKC-δ antibody. M: mitochondria; C: cytoplasm.

FIG. 3 shows a diagram of PKC-δ and its downstream effectors in DNA damage-induced apoptosis. PKC-δ is localized in the cytoplasm before the induction of apoptosis. When DNA is damaged by apoptotic stimuli, PKC-δ translocates to the nucleus, where it phosphorylates Rad9 and c-Ab1 and activates MEKK1. PKC-δ also translocates to mitochondria, where it phosphorylates PLS3 and c-Ab1, and to the Golgi, where it phosphorylates c-Ab1 and SAPK/JNK. In the plasma membrane, PKC-δ phosphorylates PLS1. The pathway from DNA damage to checkpoint activation and apoptosis is outlined.

FIG. 4 shows down-regulation of PKC-δ decreases AD198-induced apoptosis. HeLa cells were transfected with mammalian PKC-δ siRNA expression plasmid pKD-PKC-δ-v3 or a control vector. Whole cell lysates were harvested at 48 h for Western blotting. For apoptosis assays, HeLa cells at 90% confluence were transfected with pKD-PKC-δ-v3 plasmid for 48h. Cells were then treated with AD198 at 0, 1, 5, and 10 μM for 16 h. Cells were harvested and the apoptotic population was determined by PI staining. The subG₁ population was marked by M1 gate and used to represent the apoptotic population. The histogram represents the means±standard deviations from three independent experiments.

FIG. 5 shows modulation of AD198-induced apoptosis by PLS3. (a) Overexpression of PLS3 enhanced AD198-induced apoptosis. HeLa cells at 90% confluence were transfected pcDNA control vector or PLS3-expression vector for 24 h. The expression of PLS3 was shown by Western blot. Cells were then incubated with AD198 at 0, 1, 5, and 10 μM. Cells were harvested at 16 h after AD198 exposure and the apoptotic population was determined by staining with PI. The subG₁ population was marked by M1 gate and used to represent the apoptotic population. (b) Down-regulation of PLS3 by siRNA. HeLa-PLS3 cells were transfected with siRNA against PLS3 or random control siRNA at 5, 10, 15, 20 nM. Whole cell lysates were harvested at 48 h for Western blotting. Down-regulation of PLS3 abolished AD198-induced apoptosis. HeLa cells at 50% confluence were transfected with 20 nM siRNA against PLS3 or a random control siRNA for 48 h. Cells were then treated with AD198 at 0, 1, 5 and 10 μM for 16 hours followed by staining with PI. The apoptotic sub-G₁ population is marked as M1 gate and the percentages are indicated. The histogram represents the means±standard deviations from three independent experiments.

FIG. 6 shows AD198 induces PLS3 phosphorylation at threonine. (a) HeLa cells were transfected with the His-tagged PLS3 expression vector and treated with AD198 for 0, 2, 4, 6 h. Whole cell lysates were incubated with Ni affinity beads to pull down His-tagged PLS3. The pulled down samples were analyzed by Western blotting with antibodies against PT and PLS3. (b) HeLa cells were transfected with the His-tagged PLS3 vector combined with the pcDNA empty vector, PKC-δ or kinase-defective PKC-δ (PKC-δ KD) vectors. Cells were then incubated with or without AD198 (5 μM) for 2 h. Whole cell lysates were incubated with Ni beads to pull down His-tagged PLS3, and analyzed with the antibodies against PT and PLS3. (c) Down-regulation of PKC-δ suppresses AD198-induced PLS3 phosphorylation. HeLa cells were transfected with various combinations of pcDNA, pKD-PKC-δ-v3, and pCMV-His-PLS3 as indicated. Cells were treated with or without AD198 for 16 h. Whole cell lysates were probed with PKC-δ antibody, and PLS3 pulled down by Ni beads was probed with PT antibody.

FIG. 7 shows PLS3 interacts with wild-type PKC-δ but not with the inactive PKC-δ KD mutant. (a) HeLa cells expressing His-tagged PLS3 were transfected with PKC-δ or PKC-δ KD vectors. They were then incubated with AD198 for 0, 2, 4, and 6 h. The whole cell lysates were incubated with Ni beads to pull down His-tagged PLS3 and probed with antibodies against PLS3 and PKC-δ. (b) HeLa-PLS3 cells transfected with pcDNA, PKC-δ or PKC-δ KD vectors were harvested before and 2 h after AD198 treatment. PKC-δ was immunoprecipitated from whole cell lysates and analyzed with antibodies against PKC-δ and PLS3. The Western blotting of whole cell lysates revealed that the level of PLS3 expression was roughly similar before and after AD198 treatment.

FIG. 8 shows identification of Thr21 as the phosphorylation site of PLS3 induced by PKC-δ. (a) In vitro phosphorylation of recombinant PLS3 by PKC-δ. Equal amounts of recombinant PLS3 and a 6 kDa amino terminal fragment of PLS3 were mixed together and phosphorylated by purified PKC-δ in the presence of [γ-³²P]ATP. The phosphorylated sample was analyzed with SDS gel and exposed by autoradiograph. (b) PLS3 phosphorylation by PKC-δ is suppressed by T21A mutation in PLS3. Recombinant His-tagged PLS3 (wild-type) and T21A mutant was isolated by Ni beads from two elutions and analyzed with SDS gel. The upper panel shows the yield of each protein in the two-step elution by Coomassie staining. The lower panel shows the in vitro phosphorylation of equal amounts of PLS3 protein (elutes 1) by recombinant PKC-δ. (c) Subcellular fraction of cells transfected with PLS3 or PLS3(T21A) expression vectors. (d)T21A mutation abolished in vivo PLS3 phosphorylation after AD198 treatment. HeLa cells were transfected with the control vector or vectors expressing His-tagged PLS3 or PLS3(T21A). His-tagged PLS3 protein was pulled down by Ni beads from cells with or without AD198 treatment. The whole cell lysates and pulled down samples by Ni beads were analyzed with Western blotting using antibodies against PLS3 and PT. (e) Interaction of PLS3(T21A) with PKC-δ. HeLa cells were co-transfected with the PKC-δ vector and pcDNA, PLS3 or PLS3(T21A) vector, the whole cell lysates were incubated with Ni beads to pull down PLS3. The pulled down samples were analyzed with Western blotting using antibodies against PLS3 and PKC-δ. (f) HeLa cells were co-transfected as in (e), the whole cell lysates were immunoprecipitated with PKC-δ antibody. The whole cell lysates and IPs were analyzed by Western blotting with the antibody against PLS3.

FIG. 9 shows PLS3(T21A) does not enhance AD198-induced apoptosis like the wild-type PLS3. (a) HeLa cells were transfected with the empty vector or vectors expressing PLS3 or PLS3(T21A). Cells were harvested for Western blotting with antibodies against PLS3 and actin. (b) Cells then treated with DMSO or 5 μM AD198 for 16 h followed by flow cytometry analysis with PI staining. The representatives of flow cytometry analysis were shown. The sub-G₀ apoptotic population was determined and indicated in each panel. (c) Histogram to show the average of apoptotic population in control cells and cells transfected with wild-type PLS3 or PLS3(T21A). Results are the averages of three independent experiments. Statistical significance (p<0.05) was achieved by the paired t-test between wild-type PLS3 versus control or PLS3(T21A).

FIG. 10 shows AD198-induced PLS3 phosphorylation is an upstream event of caspase activation and independent of mitochondrial permeability transition.(a) Z-VAD and CsA could not block AD 198-induced loss of mitochondrial potential. HeLa cells were treated with 50 μM Z-VAD or 5 μM CsA for 30 min, and then AD198 was added at 0, 1 or 5 μM for 16 h. Cells were incubated with MitoTracker Green at 37° C. for 20 min followed by flow cytometry analysis. The histogram represents the means±standard deviations from three independent experiments. (b) Z-VAD, but not CsA, inhibits AD198-induced apoptosis. HeLa cells were treated as in (a) and collected for the apoptosis study with PI staining. (c) The means±standard deviations from three independent experiments in (b). (d) Z-VAD or CsA could not suppress AD198-induced PLS3 phosphorylation. HeLa cells were transfected with the vector expressing His-tagged PLS3. Cells were treated with AD198 (5 μM) along with Z-VAD or CsA for 2 h and harvested for Ni-bead pulldown. The whole cell lysates and the pulldown samples were probed with antibodies against PT and PLS3.

FIG. 11 shows induction of apoptosis by phosphomimetic PLS3. (a) Expression of PLS3 or PLS3(T21D). HeLa cells at 90% confluence were transfected with pcDNA control vector, PLS3- or PLS3(T21D)-expression vector for 48 h. Whole cell lysates were harvested for Western blotting using PLS3 antibody. β-actin was used as loading control. (b) Determination of apoptosis by flow cytometry analysis. HeLa cells were transfected as in (a) and then treated with 5 μM AD198 or DMSO for 16 h. Cells were harvested and stained with PI. The apoptotic population was determined by flow cytometry. The subG₁ population was marked by M1 gate and used to represent the apoptotic population. (c) Statistic analysis. The means±standard deviations of apoptotic population were calculated from three independent experiments as in (b). Statistical significance (p<0.05) was achieved by the paired t-test between PLS3(T21D) versus pcDNA control or wild-type PLS3 in DMSO groups, and between wild-type PLS3 versus pcDNA control or PLS3(T21D) in AD198 treated groups(indicated by *).

FIG. 12 shows down-regulation of PKC-δ cannot inhibit apoptosis induced by phosphomimetic PLS3. (a) Protein expression by Western blotting. HeLa cells were co-transfected with PKC-δ siRNA or pcDNA control vector and wild-type PLS3 or PLS3(T21D) vector at 90% confluence for 48 h. The down-regulation of PKC-δ and overexpression of wild-type PLS3 or PLS3(T21D) were examined by Western blotting of the whole cell lysates. (b) Determination of apoptosis by flow cytometry analysis. HeLa cells were transfected as in (a) and then incubated with or 5 μM AD198 or DMSO. Cells were harvested at 16 h later and the apoptotic population was determined by PI staining followed by flow cytometry analysis. The subG₁ population was marked by M1 gate and used to represent the apoptotic population. (c) Statistic analysis. The histogram represents the means±standard deviations from three independent experiments as in (b).

FIG. 13 shows quantification of PLS3 activity by the lipid flip-flop assay. Proteoliposomes were reconstituted with equal amount of (a) PLS3, (b) PLS3(T21A) or (c) PLS3(T21D) and analyzed with the lipid flip-flop assay. Pyrene-PC was used as the probe to quantify the rate of flip-flop. The ratio of excimer and monomer(Ie/Im) was monitored after adding either 4 mM Ca²⁺ or 80 nM tBid, followed by 4 mM Ca²⁺. The decrease of Ie/Im ratio indicates the occurrence of lipid flip-flop. Grey curves had only Ca²⁺ added at 50 seconds. Black curves had tBid added at 50 sec and Ca²⁺ added at 250 sec.

FIG. 14 shows increased tBid-binding capacity by AD198 or PLS3 phosphorylation. (a) Increased tBid-binding capacity by AD198. Mitochondria were isolated from HeLa cells treated with 5 μM AD198 or DMSO as control for 4 h. Mitochondria (50 μg by protein) were incubated with 0, 10, 20, 60 and 100 ng/μl FITC-tBid(G94E) in total volume 50 μl for 15 min followed by washing to remove unbounded probes. The fluorescent intensity was quantified with Bio-Tek microplate reader. Error bars indicate standard deviations of three independent experiments. (b) Dose response with mitochondria. Various amounts of mitochondria in (a) were incubated with 10 ng/μl tBid(G94E)-FITC as in (a). The fluorescent intensity of the washed mitochondria was quantified with Bio-Tek microplate reader. (c) Expression of PLS3 or PLS3(T21D) in selected HeLa cells. HeLa cells were transfected with pcDNA-, PLS3- or PLS3(T21D)-expression vector and selected with G418. The stable clones were subjected to subcellular fractionation for Western blotting analysis using PLS3 antibody. VADC and β-actin were used as loading control. (d) Increased tBid-binding capacity by overexpression of PLS3 or PLS3(T21D). Mitochondria were isolated from cells in (c). The tBid-binding capacity of the mitochondria from each cells were analyzed as in (a). (e) Dose response with mitochondria. Mitochondria were isolated from cells in (c) and analyzed as in (b).

FIG. 15 shows distribution of CL in the outer membrane of mitochondria. (a) HeLa cells were treated with 5 μM AD198 or DMSO as control for 4 h. Cells were stained with different concentrations of NAO and the median fluorescent intensity was determined by flow cytometry. (b) The median fluorescent intensity value of control cells stained with 35 μM NAO was considered as 100% and used to calculate the percentages in each NAO concentration. Results were average of two independent experiments. (c) After transfection for 48 h, HeLa-control, HeLa-PLS3 and HeLa-PLS3(T21D) cells were stained with different concentrations of NAO and the median fluorescent intensity was determined by flow cytometry. The median fluorescent intensity value of 35 μM NAO was considered as 100% and used to calculate the percentage in each NAO concentration. Results were average of two independent experiments.

FIG. 16 shows rescuing AD198-induced apoptosis by expression of PLS3 transgenes. (a) Western blotting analysis of PLS3 after rescuing siRNA-mediated down-regulation. PLS3* and PLS3(T21D)* represent the PLS mutants containing Gly99 (GGC→GGA), which is located at the center of PLS3 siRNA sequence. HeLa cells were transfected with scrambled control siRNA, PLS3 siRNA, PLS3, PLS3*, PLS3(T21D) or PLS3(T21D)* as indicated for 48 h before harvest. Whole cell lysates were analyzed with Western blotting using PLS3 and β-actin antibodies. (b) Apoptosis study. HeLa cells were transfected as in (a) and treated with 5 μM AD198 or DMSO only for 16 h. Apoptosis was analyzed with PI staining followed by flow cytometry analysis. The histogram represents the means±standard deviations from three independent experiments.

### DETAILED DESCRIPTION OF THE INVENTION

The disclosed method and compositions may be understood more readily by reference to the following detailed description of particular embodiments and the Example included therein and to the Figures and their previous and following description.

### A. DEFINITIONS

It must be noted that as used herein and in the appended claims, the singular forms “a ”, “an”, and “the” include plural reference unless the context clearly dictates otherwise. Thus, for example, reference to “a PKC-δ activator” includes a plurality of such activators, reference to “the cell” is a reference to one or more cells and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10”is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The terms “higher,” “increases,” “enhances,” “elevates,” or “elevation” refer to increases above control level or basal level. The terms “low,” “lower,” “inhibit,” “reduces,” or “reduction” refer to decreases below control level or basal level. For example, basal levels are normal in vivo levels prior to, or in the absence of, addition of an agent such as a PKC-δ activator.

The term “test compound” is defined as any compound to be tested for its ability to interact with PKC-δ, either directly or indirectly. Examples of PKC-δ activators include, but are not limited to, phorbol ester, PMA, mitochondrial targeting agents, and AD198.

Drugs, molecules, and compounds that come from combinatorial libraries where thousands of such ligands that are screened by drug class can be used as test compounds.

The terms “control levels” or “control cells” are defined as the standard by which a change is measured, for example, the controls are not subjected to the experiment, but are instead subjected to a defined set of parameters, or the controls are based on pre- or post-treatment levels.

Although any methods and materials similar or equivalent to those described herein can be used in the practice or testing of the present method and compositions, the particularly useful methods, devices, and materials are as described. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. GENERAL

1. Protein Kinase C

Proteasome inhibitor-induced apoptosis occurs through a mitochondrion-dependent pathway based on the increased reactive oxygen species and disruption of mitochondrial potential in proteasome inhibitor-treated cells (et al. J Biol Chem. 2003;278:33714-33723; Pei et al. Leukemia. 2003;17:2036-2045).

The protein kinase C (PKC) family consists of phospholipid-dependent protein kinases. PKC is regulated by a wide variety of metabolic pathways that involve phospholipids and calcium levels within a cell. The main regulator of the pathway is diacylglycerol (DAG), which recruits PKC to the plasma membrane and causes its activation. The activity of DAG is mimicked by the phorbol-ester tumor promoters. Once activated, PKC can phosphorylate a wide variety of cellular substrates that regulate cell proliferation and differentiation. Responses to PKC vary with the types of PKCs expressed and the types of substrates available within a cell. The PKC-δ isoform is particularly involved in the regulation of cell death. During programmed cell death, PKC-δ translocates from the cytoplasm to the nucleus, mitochondria, Golgi and plasma membrane. Several key substrates of PKC-δ in the nucleus and mitochondria have been identified, and the linkage of these PKC-δ targets to regulation of DNA damage checkpoints and mitochondrial apoptosis has helped establish the mechanism of PKC-δ-induced apoptosis.

Involvement of PKC-δ in apoptosis has been demonstrated by activation of PKC-δ by a variety of apoptotic stimuli, including TNF-α the Fas ligand, UV and γ irradiation, and etoposide treatment. Activated PKC-δ translocates from cytoplasm to various organelles during apoptosis. Several critical substrates of PKC-δ have been identified, including nuclear Rad9 (Yoshida et al. Embo J. 2003;22:1431-1441), mitochondrial Ab1 kinase (Sun et al. J Biol Chem. 2000;275:7470-7473) and phospholipid scramblase 3 (Liu et al. Cancer Res. 2003;63:1153-156). Inhibition of PKC-δ by rottlerin, or by a dominant negative mutant results in suppression of apoptosis. Therefore, PKC-δ is an important mediator for apoptosis.

Regulation of PKC-δ activity is mediated by at least three mechanisms. The regulatory C1 domain has an inhibitory effect on the catalytic domain found at the carboxyl terminus. One way to release inhibition is by interaction of diacylglycerol or phorbol esters with the C1 domain, which triggers a conformational change. A second mechanism is mediated by cleavage of the catalytic domain from the C1 regulatory domain, which is achieved during apoptosis by activated caspase 3. Tyrosine phosphorylation of PKC-δ at Tyr64 and 187 is essential for the cleavage and the apoptotic effect of PKC-δ. Tyr311 phosphorylation by Lck kinase after H₂O₂ treatment enhances basal PKC-δ activity and elevates its maximal activity in the presence of diacylglycerol. Translocation of PKC-δ to mitochondria was shown in U937 myeloid leukemia cells and keratinocytes. The translocation can be induced by phorbol ester and oxidative stress. With UV irradiation, mitochondrial targeted PKC-δ was cleaved by caspase 3 to generate the active catalytic fragment of PKC-δ. Finally, activated PKC-δ undergoes ubiquitination and degradation through the proteasome pathway, which prevents a persistent effect of PKC-δ. Hence, stabilization of PKC-δ can be a mechanism of proteasome inhibitor-induced mitochondrial dysfunction.

Co-incubation of bortezomib with a PKC-δ inhibitor, rottlerin, suppresses bortezomib-induced apoptosis in U937 cells (Example 1). Western analysis of U937 cells treated with bortezomib revealed accumulation of full-length PKC-δ in the first 4 hours. By 16 hours an active catalytic fragment of PKC-δ accumulated in mitochondria. The cleavage of PKC-δ after bortezomib treatment was mediated by caspases, because a pan-caspase inhibitor BAF prevented the appearance of the active fragment of PKC-δ.

As disclosed herein, accumulation of the active PKC-δ fragment in mitochondria is responsible for proteasome inhibitor-induced mitochondrial damage. Because PKC-δ is essential to achieve the full effect of proteasome inhibition, enhancing or increasing the level of PKC-δ in the cell, or enhancing mechanisms and pathways associated with the efficient processing and action of PKC-δ, can create a synergistic or additive effect when used with a proteasome inhibitor, such as bortezomib. Failure of activation of PKC-δ can also be a mechanism of resistance to the proteasome inhibitor. Therefore, a combination of a proteasome inhibitor and a PKC-δ activator can be used to overcome proteasome inhibitor resistance and to avoid the development of drug resistance to proteasome inhibitor in cancer, as well as to create a synergistic or additive effect in cancer management.

2. Phospholipid Scramblase 3

Phospholipid scramblases (PLS) are enzymes responsible for bidirectional movement of phospholipids between two lipid compartments (Bevers et al. Biochim Biophys Acta, 1439:317-330 (1999), Williamson and Schlegel Biochim Biophys Acta 1585:531-63 (2002)). Four PLS family members have been identified by genome analysis (Wiedmer et al. Biochim Biophys Acta, 1467:244-253 (2000)). PLS1 is in the plasma membrane and can translocate to nucleus through the importin pathway (Wiedmer et al. Biochemistry 42:1227-1233 (2003), Ben-Efraim et al. Biochemistry 43:3518-3526 (2004)). PLS2 is in the nucleus but less well characterized (Yu et al. J Biol Chem, 278:9706-9714 (2003)). PLS3 is present in mitochondria (Liu et al. Mol Cancer Res 1:892-902 (2003), Liu et al. Cancer Res 63:1153-1156 (2003)). It has been shown that PLS3 is a critical regulator of mitochondrial structure, respiratory function, and distribution of cardiolipin (Liu et al. (2003)). Overexpression of wild-type PLS3 enhanced sensitivity to UV- and tBid-induced apoptosis, and increased the amount of cardiolipin in the mitochondrial outer membrane. In contrast, mitochondria with expression of an inactive PLS3 mutant that abolished the calcium-binding motif had lower amounts of cytochrome c and cardiolipin, and exhibited densely packed cristae. Consequently their respiratory function determined by oxygen consumption was significantly compromised (Liu et al. (2003)). Consistent with the respiratory defect in cells with suppressed PLS3 function, mice with targeted deletion of PLS3 had insulin resistance, glucose intolerance and dyslipidemia that lead to aberrant accumulation of abdominal fat (Wiedmer et al. Proc Natl Acad Sci USA 101:13296-13301 (2004)).

In each organelle, PKC-δ phosphorylates different substrates to induce various downstream events that eventually lead to cell death (Liu et al. (2003)). One nuclear substrate is Rad9, which forms the 9-1-1 complex with Hus1 and Rad1 to regulate DNA damage response (Yoshida et al. (2003), Volkmer and Karnitz J Biol Chem 274:567-570 (1999)). A substrate in the plasma membrane is PLS1 (Frasch et al. J Biol Chem 275:23065-23073 (2000)). Based on the observation that PLS1 is a substrate for PKC-δ, it was established that PLS3 is a physiologic target of PKC-δ when PKC-δ translocates to mitochondria. After UV irradiation, PKC-δ physically interacted with and phosphorylated PLS3 with high affinity. Cells expressing wild-type PLS3 (HeLa-PLS3) became apoptotic upon phorbol ester stimulation, whereas the control cells did not. Expression of a mitochondrion-targeted PKC-δ enhanced apoptosis more prominently in HeLa-PLS3 cells than control HeLa cells and HeLa cells expressing an inactive PLS3 mutant.

With the establishment of PKC-δ as a death-promoting kinase, a strategy to induce apoptosis is to develop an activator of PKC-δ. This has been achieved by the development of the extranuclear-targeted anthracycline derivatives, N-benzyladriamycin-14-valerate (AD198) and N-benzyladriamycin-14-pivalate (AD445). The antitumor activity of AD198 is superior to that of doxorubicin, suggesting a distinct mechanism of cytotoxicity (Roaten et al. (2001), Minotti et al. Pharmacol Rev 56:185-229 (2004)). AD198 rapidly accumulates in the cytoplasm and is able to circumvent resistance due to expression of multidrug resistance protein ( Lothstein et al. (1992), Lothstein et al. Anticancer Drugs 5:623-633 (1994), Lothstein et al. Anticancer Drugs 9:58-66 (1998)). Moreover, AD198 can override the anti-apoptotic function of Bcl-2 and circumvent both NF-κB and Bcl-xL-mediated resistance (Barrett et al. (2002), Bilyeu et al. Mol Pharmacol 65:1038-1047 (2004)). The C1b regulatory domain of PKC was demonstrated to be the molecular target of AD198 (Roaten et al. (2001), Roaten et al. (2002)). AD198 promotes rapid translocation and activation of PKC-δ to mitochondria, which leads to cytochrome c release and caspase activation (Roaten et al. (2002), Minotti et al. (2004)). In example 3, the interaction of PLS3 and PKC-δ in response to AD198 is shown, and it is demonstrated that PLS3 is phosphorylated at Thr21 by PKC-δ.

Apoptosis, PKC-δ and PLS3

Programmed cell death or apoptosis is a fundamental feature of many biological processes (Evan et al. Science 281:1317-1322 (1998); Green et al. Cancer Cell 1:19-30 (2002)). It is characterized by distinct biochemical and morphological changes in cells, including nuclear fragmentation, chromatin condensation and membrane blebbing (Kerr et al. Br J Cancer 26:239-257 (1972)). The extrinsic pathway of apoptosis is activated by death domain receptors, forming a death-induced signaling complex (DISC) to transduce cell death signals to the nucleus and mitochondria (Krammer, P. H. Nature 407:789-795 (2000), Baud and Karin Trends Cell Biol 11:372-377 (2001)). During apoptosis the plasma membrane itself, although maintaining its integrity, exhibits phospholipid translocation. Phosphatidylserine (PS) is translocated from the inner to outer leaflet of the plasma membrane to serve as a signal for recognition and clearance of apoptotic cells by macrophages (Fadok et al. J Immunol 148:2207-2216 (1992)). This process is a complicated event regulated by at least three enzymes, phospholipid scramblase (PLS), aminophospholipid translocase (flippase) and floppase, which are responsible for phospholipid translocation in the inward or outward direction (Bevers et al. Biol Chem 379: 973-986 (1998), Bevers et al. Biochim Biophys Acta 1439:317-330 (1999)).

In the intrinsic apoptotic pathway, mitochondria are the most important organelles, and the integrators of apoptosis (Wang et al. Genes Dev 15:2922-2933 (2001)). Many critical mediators of apoptosis are either localized in or translocated to mitochondria to regulate the release of various apoptogenic factors, such as cytochrome c (Li et al. Cell 91: 479-489 (1997)), SMAC ( Verhagen et al. [In Process Citation]. Cell 102:43-53 (2000), Du et al. [In Process Citation]. Cell 102:33-42 (2000)), endonuclease G (Li et al. Nature 412:95-99 (2001), Parrish et al. Nature 412: 90-94 (2001)), and apoptosis-inducing factor (AIF) (Susin et al. Nature 397:441-446 (1999)), to induce various downstream apoptotic responses. In addition to protein factors, changes in mitochondrial lipids were also recognized (McMillin and Dowhan Biochim Biophys Acta 1585:97-107 (2002), Cristea et al. Chem Phys Lipids 129:133-160 (2004)). One distinct component of mitochondrial lipids is cardiolipin (CL), which is a phospholipid synthesized and localized exclusively in mitochondria (Schlame et al. Prog Lipid Res 39:257-288 (2000)). Changes in CL have been demonstrated in early steps of apoptosis, such as translocating to the outer leaflet of the mitochondrial inner membrane (Garcia Fernandez et al. Cell Growth Differ 13:449-455 (2002)), and eventually to the surface of plasma membrane (Sorice et al. Cell Death Differ 11:1133-1145 (2004)). CL is also responsible for recruitment of pro-apoptotic proteins tBid to mitochondria (Lutter et al. Nat Cell Biol 2:754-761(2000)) and to activate Bax and induce the release of apoptogenic proteins (Luo et al. Cell 94:481-490 (1998), Eskes et al. Mol Cell Biol 20:929-935 (2000), Desagher et al. J Cell Biol 144:891-901 (1999), Korsmeyer et al. Cell Death Differ 7:1166-1173 (2000)). In mitochondria, CL forms a supramolecular complex with tBid and Bax (Kuwana et al. Cell 111:331-342 (2002)). If CL was depleted in cells expressing the temperature sensitive mutant of the CL synthesis enzyme, Bid could not be targeted to mitochondria (Lutter et al. (2000)). One enzyme that may play a role in regulating CL localization in mitochondria is phospholipid scramblase 3 (PLS3), which plays important roles in mitochondrial morphology, respiratory function and apoptotic response (Liu et al. Mol Cancer Res 1:892-902 (2003)). It has been shown that cells with overexpression of PLS3 were more sensitive to UV and TNF-α-induced apoptosis, whereas cells expressing an inactive PLS3 mutant were not. Overexpression of PLS3 increases the amount of CL in the mitochondrial outer membrane, which can enhance the targeting of tBid to mitochondria to induce cytochrome c and SMAC release (Liu et al. (2003)).

Regulation of PLS3 activity could be mediated by post-translational phosphorylation (Liu et al. Cancer Res 63:1153-1156 (2003)). It has been established that PLS3 is a physiological target of PKC-δ-induced apoptosis in mitochondria. PKC-δ can physically interact with and phosphorylate PLS3 with a high affinity. Mutation of Thr21 in PLS3 to alanine abolished phosphorylation of PLS3 by PKC-δ in vitro and diminished the interaction between PLS3 and PKC-δ. A PKC-δ activator, N-benzyladriamycin-14-valerate (AD198), induced PLS3 phosphorylation in vivo. Expression of wild type PLS3, but not the PLS3(T21A) mutant, significantly enhanced AD198-induced apoptosis. These studies show that phosphorylation of PLS3 at Thr21 by PKC-δ is a mechanism of PLS3 activation during apoptosis.

### C. MATERIALS

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if bortezomib or another proteasome inhibitor, is disclosed and discussed and a number of modifications that can be made to a number of molecules including the bortezomib or another proteasome inhibitor are discussed, each and every combination and permutation of bortezomib or another proteasome inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, as an example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A proteasome inhibitor is any substance which directly or indirectly inhibits the 20S or 26S proteasome or the activity thereof. Preferably, such inhibition is specific, i.e., the proteasome inhibitor inhibits proteasome activity at a concentration that is lower than the concentration of the inhibitor required to produce another, unrelated biological effect. Preferably, the concentration of the proteasome inhibitor required for proteasome inhibition is at least 2-fold lower, more preferably at least 5-fold lower, even more preferably at least 10-fold lower, and most preferably at least 20-fold lower than the concentration required to produce an unrelated biological effect.

Non-limiting examples of proteasome inhibitors for use in the present invention include peptide aldehydes (see, e.g., Stein et al. WO 95/24914 published Sep. 21, 1995; Siman et al. WO 91/13904 published Sep. 19, 1991; Iqbal et al. J. Med. Chem. 38:2276-2277 (1995)), vinyl sulfones (see, e. g., Bogyo et al., Proc. Natl. Acad. Sci. 94:6629 (1997)), [agr]′[bgr]′-epoxyketones (see, e.g., Spaltenstein et al. Tetrahedron Lett. 37:1343 (1996)); peptide boronic acids (see, e.g., Adams et al. WO 96/13266 published May 9, 1996; Siman et al. WO 91/13904 published Sep. 19, 1991), and lactacystin and lactacystin analogs (see, e.g., Fenteany et al. Proc. Natl. Acad. Sci. USA 94:3358 (1994); Fenteany et al. WO 96/32105 published Oct. 19, 1996), each of which is hereby incorporated by reference in its entirety, an at least for material related to proteosome inhibitors and their structures. Also included by reference in their entirety are U.S. Pat. Nos. 6,713,446, 6,699,835, 6,747,150, 6,617,317, 6,613,541, 6,548,668, 6,465,433, 6,297,217, 6,294,560, 6,271,199, 6,133,308, 6,117,887, 6,083,903, 5,834,487, 5,780,454, 5,693,617, and 6,066,730 for their teaching of proteasome inhibitors and their structures.

The synthesis of N-terminal peptidyl boronic ester and acid compounds, in general and of specific compounds, has been described previously (Shenvi et al. U.S. Pat. No. 4,499,082 issued Feb. 12, 1985; Shenvi et al. U.S. Pat. No. 4,537,773 issued Aug. 27, 1985; Siman et al. WO 91/13904 published Sep. 19, 1991; Kettner et al., J. Biol. Chem. 259(24): 15106-15114 (1984). These compounds have been shown to be inhibitors of certain proteolytic enzymes (Shenvi et al. U.S. Pat. No. 4,499,082 issued Feb. 12, 1985; Shenvi et al. U.S. Pat. No. 4,537,773; Siman et al. WO 91/13904 published Sep. 19, 1991; Kettner at al., J. Biol. Chem. 259(24):15106-15114 (1984). A class of N-terminal tri-peptide boronic ester and acid compounds has been shown to inhibit the growth of cancer cells (Kinder et al. U.S. Pat. No. 5,106,948 issued Apr. 21, 1992). A broad class of N-terminal tri-peptide boronic ester and acid compounds and analogs thereof has been shown to inhibit renin (Kleeman et al. U.S. Pat. No. 5,169,841 issued Dec. 8, 1992).

In the cell, there is a soluble proteolytic pathway that requires ATP and involves covalent conjugation of the cellular proteins with the small polypeptide ubiquitin (“Ub”) (Hershko et al., A. Rev. Biochem. 61:761-807 (1992); Rechsteiner et al., A. Rev. Cell. Biol. 3:1-30 (1987)). Thereafter, the conjugated proteins are hydrolyzed by a 26S proteolytic complex containing a 20S degradative particle called the proteasome (Goldberg, Eur. J. Biochem. 203:9-23 (1992); Goldberg et al., Nature 357:375-379 (1992)). This multicomponent system is known to catalyze the selective degradation of highly abnormal proteins and short-lived regulatory proteins.

The 20S proteasome is composed of about 15 distinct 20-30 kDa subunits. It contains three different peptidase activities that cleave specifically on the carboxyl side of the hydrophobic, basic, and acidic amino acids (Goldberg et al., Nature 357:375-379 (1992); Goldberg, Eur. J. Biochem. 203:9-23 (1992); Orlowski, Biochemistry 29:10289 (1990); Rivett et al., Archs. Biochem. Biophys. 218:1 (1989); Rivett et al., J. Biol. Chem. 264:12,215-12,219(1989); Tanaka et al., New Biol. 4:1-11 (1992)). These peptidase activities are referred to as the chymotrypsin-like activity, the trypsin-like activity, and the peptidylglutamyl hydrolyzing activity, respectively.

Various inhibitors of the peptidase activities of the proteasome have been reported (Dick et al., Biochemistry 30:2725-2734 (1991); Goldberg et al., Nature 357:375-379 (1992); Goldberg, Eur. J. Biochem. 203:9-23 (1992); Orlowski, Biochemistry 29:10289 (1990); Rivett et al., Archs. Biochem. Biophys. 218:1 (1989); Rivett et al., J. Biol. Chem. 264:12,215-12,219 (1989); Tanaka et al., New Biol. 4:1-11 (1992); Murakami et al., Proc. Natl. Acad. Sci. U.S.A. 83:7588-7592 (1986); Li et al., Biochemistry 30:9709-9715 (1991); Goldberg, Eur. J. Biochem. 203:9-23 (1992); Aoyagi et al., Proteases and Biological Control, Cold Spring Harbor Laboratory Press (1975), pp. 429-454, all of which are incorporated at least for material related to proteosome inhibitors and their structures.

Bortezomib (also PS-341 or Velcade®) is an inhibitor of the 26S proteasome developed as an anti-neoplastic agent (LeBlanc et al. Cancer Res. 2002;62:4996-5000).

Bortezomib is a modified dipeptidyl boronic acid. The product is provided as a mannitol boronic ester which, in reconstituted form, consists of the mannitol ester in equilibrium with its hydrolysis product, the monomeric boronic acid. The drug substance exists in its cyclic anhydride form as a trimeric boroxine. The chemical name for bortezomib, the monomeric boronic acid, is [(1R)-3-methyl-1-[[(2S)-1-oxo-3-phenyl-2-[(pyrazinylcarbonyl)amino]propyl]amino]butyl] boronic acid. The molecular weight is 384.24. The molecular formula is: C₁₉H₂₅BN₄O₄. The solubility of bortezomib, as the monomeric boronic acid, in water is 3.3-3.8 mg/mL in a pH range of 2-6.5.

Several compounds including, but not limited to, MG-115, MG-132, and NLVS (Calbiochem) are analogs to bortezomib and can function as equivalents, as they are able to block proteasomes as well. Proteasomes play an important role in proliferation and cell cycle control (Mitsiades et al. Proc Natl Acad Sci USA. 2002;99:14374-14379). Many critical proteins, including p53, p21 and p27 cyclin-dependent kinase inhibitors, are regulated by proteasome-dependent proteolysis (An et al. Leukemia. 2000;14:1276-1283). Blocking proteasomes leads to accumulation of these proteins, which in turn blocks the cell cycle and triggers apoptosis. Because these proteins are crucial steps in tumor formation, the proteasome pathway is a logical target for therapeutic intervention.

Blocking the proteasome pathway has another anti-neoplastic activity as well, since the transcriptional activator NF-κB (Palombella et al. Cell. 1994;78:773-785) is also regulated by this pathway. NF-κB is sequestered in the cytoplasm by an inhibitor IκB, which undergoes proteasome-dependent proteolysis upon mitogenic stimulation. Without IκB, NF-κB translocates to the nucleus and induces transcriptional activation of multiple genes involved in cell adhesion and cancer metastasis (Read et al. Immunity. 1995;2:493-506). Survival factors such as Bcl-2 and IL-6 are also activated. Elevated NF-kB and bc1-2 activities allow cancer cells to defend themselves against treatment with standard chemotherapy agents. However, when proteasomes are inhibited, IκB is stabilized and blocks NF-κB and expression of these survival factors, thereby acting as an anti-neoplastic agent. Therefore, by blocking the normal function of NF-kB and bc1-2, a proteasome inhibitor can cause the death of cancer cells.

“PKC-δ activators” are referred to throughout the specification. A “PKC-δ activator” is defined as a substance capable of enhancing, increasing, or upregulating the presence or availability or one or more activities of PKC-δ in the cell. PKC-δ can be activated in multiple ways, either directly or indirectly. Examples of PKC-δ activators include substances that upregulate production of PKC-δ. Because PKC-δ is involved in the regulation of cell death (apoptosis), any substance that activates PKC-δ, thereby causing apoptosis (directly or indirectly) is considered a PKC-δ activator. Several key substrates of PKC-δ in the nucleus and mitochondria have been identified, and the linkage of these PKC-δ targets to regulation of DNA damage checkpoints and mitochondrial apoptosis has helped establish the mechanism of PKC-δ induced apoptosis. Therefore, a substance that increases localization of PKC-δ to mitochondria in the presence of bortezomib is also considered a PKC-δ activator.

Another example of a PKC-δ activator is a substance that releases inhibition of PKC-δ. The regulatory C1 domain contains a pseudosubstrate site and has an inhibitory effect on the catalytic domain found at the carboxyl terminus. One way to release inhibition is by interaction of diacylglycerol or phorbol esters, for example, with the C1 domain, which triggers a conformational change and moves the pseudosubstrate site out of the catalytic subunit.

Another type of PKC-δ activator is a substance that increases cleavage of PKC-δ. It has been demonstrated that PKC-δ is a substrate for caspase-3, which cleaves PKC at the DMQD³³⁰N site in the hinge region, generating a 40-kDa C-terminal fragment known as the catalytic domain. In contrast to calpain or proteasome, which leads to degradation of PKC-δ, caspases cause only limited proteolysis. The separation of the autoinhibitory regulatory domain from the catalytic domain results in its activation.

Tyrosine phosphorylation of PKC-δ at Tyr64 and 187 is essential for the cleavage and the apoptotic effect of PKC-δ. Therefore, another PKC-δ activator is a substance that enhances tyrosine phosphorylation of PKC-δ, which in turn increases cleavage of PKC-δ. Tyr311 phosphorylation by Lck kinase after H2O2 treatment enhances basal PKC-δ activity and elevates its maximal activity in the presence of diacylglycerol. Translocation of PKC-δ to mitochondria was shown in U937 myeloid leukemia cells and keratinocytes. The translocation can be induced by phorbol ester and oxidative stress, for example. With UV irradiation, mitochondrial targeted PKC-δ was cleaved by caspase 3 to generate the active catalytic fragment of PKC-δ. Another embodiment includes a substance that blocks Src kinase, thereby activating PKC-δ , because Src tyrosine kinase phohsphorylates PKC-δ at several sites and tyrosine phosphorylation of PKC-δ results in PKC-δ inactivation. Therefore, Src kinase is involved in the modulation of tyrosine phosphorylation of PKC-δ.

Another example of a PKC-δ activator is a substance that blocks PKC-δ degradation. Without the degradation of PKC-δ, the action of activated PKC-δ leads to cell death. In order for cells to prevent this from happening, cells utilize the proteasome degradation pathway to downregulate the level and activity of PKC-δ. Since activated PKC-δ undergoes ubiquitination and degradation through the proteasome pathway, which prevents a persistent effect of PKC-δ, an activator of PKC-δ can be a substance that blocks, prevents, or decreases PKC-degradation.

Also disclosed are PKC-δ pathway activators. As discussed herein there are a number of molecules that are involved in the PKC-δ signaling pathways. A PKC-δ pathway activator is a molecule that activates a molecule which is in the PKC-δ signaling pathway.

1. Combination Compositions

One finding disclosed herein is that a beneficial composition is one which comprises a proteasome inhibitor, such as Bortezomib, as well as a PKC-δ activator or a PKC-δ pathway activator. Thus disclosed are compositions comprising Bortezomib and/or its functional analogs and another therapeutic molecule wherein the other therapeutic molecule comprises a PKC-δ activator and/or a PKC-δ pathway activator.

AD 198, a derivative of adriamycin, can kill cancer cells by activating PKC-δ. This compound can have a synergistic affect with the proteasome inhibitor, and can be used alone or in combination with the proteasome inhibitor in the methods disclosed herein. Proteasome inhibitors also have synergistic effects with several chemotherapeutic agents, including radiation, irinotecan, gemcitabine in lung cancer, and pancreatic cancer.

Examples of proteasome inhibitors include, in addition to the general class of compounds described above, MG-115 (carbobenzoxyl-L-leucyl-L-leucyl-L-norvalinal), MG132 (carbobenzoxyl-L-leucyl-L-leucyl-L-leucinal), and NVLS (4-hydroxy-5-iodo-3-nitrophenylacetyl=Leu-Leu-Leu-vinylsulfone). These compounds are peptidyl derivatives that mimic the peptidyl substrate of proteasomes and are useful with the methods disclosed herein.

Examples of PKC-δ pathway activators that can be used in combination with the disclosed compositions are Src kinase inhibitors, such as PD173958 (J. Biol. Chem. 277: 12318) and CGP76030 (Nat Genet. 36: 440).

2. Homology/Identity

Reference to sequences for PKC-δ and its fragments are found throughout the specification. It is understood that one way to define any known variants and derivatives or those that might arise, of the disclosed genes and proteins herein is through defining the variants and derivatives in terms of homology to specific known sequences. For example SEQ ID NO: 2 sets forth a particular nucleic acid sequence of mRNA of PKC-δ and SEQ ID NO: 1 sets forth a particular sequence of the protein encoded by SEQ ID NO: 2, the PKC-δ protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

3. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

4. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

5. Nucleic Acids

There are a variety of molecules disclosed herein that are nucleic acid based, including for example the nucleic acids that encode PKC-δ as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to, for example, SEQ ID NO: 1 as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

iii. Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

iv. Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of PKC-δ or the genomic DNA of PKC-δ or they can interact with the polypeptide of PKC-δ. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹² . Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al. Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

6. Peptides

i. Protein Variants

As discussed herein there are numerous variants of the PKC-δ protein and other proteins that are known and herein contemplated. In addition, to the known functional PKC-δ variants there are derivatives of the PKC-δ proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala A |
| Allosoleucine | AIle |
| Arginine | Arg R |
| asparagines | Asn N |
| aspartic acid | Asp D |
| Cysteine | Cys C |
| glutamic acid | Glu E |
| Glutamine | Gln Q |
| Glycine | Gly G |
| Histidine | His H |
| Isolelucine | Ile I |
| Leucine | Leu L |
| Lysine | Lys K |
| Phenylalanine | Phe F |
| praline | Pro P |
| pyroglutamic | PGlu |
| acid | |
| Serine | Ser S |
| Threonine | Thr T |
| Tyrosine | Tyr Y |
| Tryptophan | Trp W |
| Valine | Val V |

TABLE 2
Amino Acid Substitutions
Original Residue Exemplary Conservative Substitutions, others are
known in the art.

Ala/ser
Arg/lys, gln
Asn/gln; his
Asp/glu
Cys/ser
Gln/asn, lys
Glu/asp
Gly/pro
His/asn; gln
Ile/leu; val
Leu/ile; val
Lys/arg; gln;
Met/Leu; ile
Phe/met; leu; tyr
Ser/thr
Thr/ser
Trp/tyr
Tyr/trp; phe
Val/ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 2 sets forth a particular sequence of PKC-δ mRNA, and SEQ ID NO: 1 sets forth a particular sequence of a PKC-δ protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzynol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 1 is set forth in SEQ ID NO: 2.

It is understood that while no amino acid sequence indicates what particular DNA sequence encodes that protein within an organism, where particular variants of a disclosed protein are disclosed herein, the known nucleic acid sequence that encodes that protein in the particular PKC-δ from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO-(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### D. METHODS

1. Methods of Treatment

Disclosed are methods of treating a subject comprising administering to the subject an effective amount of a proteasome inhibitor, such as bortezomib, and a PKC-δ activator or PKC-δ pathway activator, as discussed herein. A proteasome inhibitor, such as bortezomib, and PKC-δ can be used in the treatment of cancer, for example. However, the compositions can be used to treat any disease where uncontrolled cellular proliferation occurs. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general, such as B cell lymphoma, T cell lymphoma, mycosis fingoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. Bortezomib has been shown to be effective in the treatment of multiple myelomas, for example.

Examples of PKC-δ activators include, but are not limited to, phorbol ester, PMA, and AD198.

2. Methods of Detection

Also disclosed are methods of detecting activation of PKC-δ comprising detecting phosphorylation of PLS3, wherein a higher level of PLS3 compared to a control indicates activation of PKC-δ. This method can be used in vitro, ex vivo, and in vivo. This method can be used with a substance that activates PKC-δ, or with a test substance to determine if it activates PKC-δ. It can be used to test multiple substances together as well, to determine if they interact with each other in a way that increases or decreases phosphorylation of PKC-δ.

For example, when a subject is treated with an agent that induces PKC-δ to treat a tumor, one can determine whether the agent is effective in reducing the tumor using this method. This allows for the determination of why the drug didn't work if it was not effective in reducing the tumor. When a drug is not effective, there are at least two major possibilities. One is that the drug works by achieving its desired effect, for examples activating PKC-δ, but the tumor is not affected due to other mechanisms that prevent PKC-δ-induced cell death. Another possibility is that the drug did not reach tumor, or the tumor develops mechanisms to prevent the drug from acting, i.e., activating PKC-δ. Determining the cause of the drug's ineffectiveness allows for refinement during the drug development process. A surrogate marker, such as PLS3, allows one to determine whether the drug has achieved the desired effect such as PKC-δ activation, and if not, why.

Tumor specimens, for example, can be used to test the phosphorylation of PLS3. If PLS3 becomes more phosphorylated, it represents that the tested PKC-δ activator is effective. If, on the contrary, PLS3 is not phosphorylated, the drug is not effective, and the reason why can be determined.

3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

The proteasome inhibitor can be given by injection into a vein (intravenously) through a cannula (a fine tube inserted into the vein). It can also be given through a central line which is inserted under the skin into a vein near the collarbone, or through a PICC line which is inserted into a vein in the crook of the arm.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

i. Pharmaceutically Acceptable Carriers

The compositions, including PKC-δ activator and/or PKC-δ pathway activator, can be used therapeutically in combination with a proteasome inhibitor, and additionally can be used with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

ii. Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drags are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1μg/kg to up to 100 mg/kg of body weight or more per day, depending on the factors mentioned above.

The proteasome inhibitor can be given as four doses over a three-week period, for example. The doses can be given on the first and fourth day of the first two weeks, followed by a ten-day rest period. This completes one cycle of treatment. Under the situation of adverse effect induced by the proteasome inhibitor, it can be reduced to weekly rather than twice a week.

A proteasome inhibitor can be given at less than 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 4.5, 5.0, 10.0, or 20.0 mg/m² or greater. The proteasome inhibitor can be given twice a day or more, once a day, once every other day, twice a week, once a week, once every two weeks, once a month, or once only, or any amount greater than, less than, or in between these specified ranges.

Following administration of a disclosed composition, such as a PKC-δ activator and/or PKC-δ pathway activator, for treating, inhibiting, or preventing cancer, for example, the efficacy of the PKC-δ activator and/or PKC-δ pathway activator can be assessed in various ways well known to the skilled practitioner. For instance, one of ordinary skill in the art will understand that a composition, such as a PKC-δ activator and/or PKC-δ pathway activator, disclosed herein is efficacious in treating or inhibiting cancer in a subject by observing that the composition causes apoptosis. Apoptosis can be measured by methods that are known in the art, for example, such as (1) fluorescent dye JC-1 to measure mitochondrial potential, (2) TUNEL assay (terminal deoxynucleotidyl transferase mediated dUTP-biotin nick end labeling) (3) MTT assay (4) annexin V binding and (5) DNA fragmentation study. Efficacy of the administration of the disclosed composition may also be determined by measuring the activity of PKC-δ.

4. Methods of Screening

Disclosed are methods of identifying both therapeutic targets as well as drugs or molecules that can modulate those and existing targets. Disclosed herein is the fact that bortezomib and molecules that function like bortezomib require PKC-δ to achieve their fall therapeutic effect. Furthermore, also disclosed is that bortezomib and molecules that function like bortezomib increase not only the amount of PKC-δ, but also the amount of active PKC-δ in cells. This knowledge can be used to identify molecules that function with bortezomib as anti-cell proliferative compositions, increasing in effect the therapeutic power of bortezomib. Thus, methods that screen for activators of PKC-δ in the presence of bortezomib or molecules that function like bortezomib are desirable molecules. Many different types of screening methods can be used, for example, methods that utilize cells, methods that use in vitro assays, and methods that use binding assays, for example. There numerous embodiments disclosed herein, but it is understood that any combination of the disclosed steps and assays disclosed herein can be used to identify molecules understood as activators of PKC-δ and/or molecules that function to increase the therapeutic effect of bortezomib.

Also disclosed are methods of administering bortezomib and its functional and structural equivalents in conjunction with molecules considered to be PKC-δ activators and/or PKC-δ pathway activators or agonists, such as

Also disclosed are methods of identifying a substance that can act as a PKC-δ activator in the presence of a proteasome inhibitor. For example, disclosed are methods of identifying a substance that upregulates production of PKC-δ in the presence of a proteasome inhibitor, comprising: a) exposing cells expressing PKC-δ to a proteasome inhibitor; b) exposing the cells of step a) to a test substance; c) measuring the level of PKC-δ in the cells of step b); and c) comparing the level of PKC-δ in the cells of step c) to a control level, wherein a higher level of PKC-δ indicates a substance that upregulates production of PKC-δ in the presence of a proteasome inhibitor.

Also disclosed is a method of identifying a substance that increases localization of PKC-δ to mitochondria in the presence of a proteasome inhibitor, comprising a) exposing cells expressing PKC-δ to a proteasome inhibitor; b) exposing the cells of step a) to a test substance; c) measuring the level of PKC-δ in the cells of step b); and d) comparing the level of PKC-δ in the cells of step c) to a control level, wherein a higher level of PKC-δ indicates a substance that increases localization of PKC-δ to mitochondria in the presence of a proteasome inhibitor.

Also disclosed is a method of identifying a substance that increases cleavage of PKC-δ in the presence of a proteasome inhibitor, comprising: a) exposing cells expressing PKC-δ to a proteasome inhibitor; b) exposing the cells of step a) to a test substance; c) measuring the level of cleaved PKC-δ in the cells of step b); and d) comparing the level of cleaved PKC-δ in the cells of step c) to a control level, wherein a higher level of cleaved PKC-δ indicates a substance that increases cleavage of PKC-δ in the presence of a proteasome inhibitor.

Also disclosed is a method of identifying a substance that blocks PKC-δ degradation in the presence of a proteasome inhibitor, comprising: a) exposing cells expressing PKC-δ to a proteasome inhibitor; b) exposing the cells of step a) to a test substance; c) measuring the level of PKC-δ degradation in the cells of step b); and d) comparing the level of degraded PKC-δ in the cells of step c) to a control level, wherein a lower level of degraded PKC-δ indicates a substance that blocks degradation of PKC-δ in the presence of a proteasome inhibitor.

Also disclosed is a method of identifying a substance that enhances tyrosine phosphorylation of PKC-δ in the presence of a proteasome inhibitor, comprising: a) exposing cells expressing PKC-δ to a proteasome inhibitor; b) exposing the cells of step a) to a test substance; c) measuring the level of phosphorylated PKC-δ in the cells of step b); and d) comparing the level of phosporylated PKC-δ in the cells of step c) to a control level, wherein a higher level of phosporylated PKC-δ indicates a substance that enhances tyrosine phosphorylation of PKC-δ in the presence of a proteasome inhibitor.

Also disclosed is a method of identifying a substance that blocks Src kinase, thereby activating PKC-δ, in the presence of a proteasome inhibitor, comprising: a) exposing cells expressing PKC-δ to a proteasome inhibitor; b) exposing the cells of step a) to a test substance; c) measuring the level of Src kinase in the cells of step b); and d) comparing the level of Src kinase in the cells of step c) to a control level, wherein a lower level of Src kinase indicates a substance that blocks Src kinase in the presence of a proteasome inhibitor.

i. Combinatorial Chemistry

The disclosed compositions can be used as targets for any combinatorial technique to identify molecules or macromolecular molecules that act as PKC-δ activators. Also disclosed are the compositions that are identified through combinatorial techniques or screening techniques described herein.

It is understood that when using the disclosed compositions in combinatorial techniques or screening methods, molecules, such as macromolecular molecules, will be identified that have particular desired properties such as inhibition or stimulation of the target molecule's function. The molecules identified are also disclosed.

It is understood that the disclosed methods for identifying molecules that enhance the activity of, for example, PKC-δ, can be performed using high through put means. For example, putative inhibitors can be identified using Fluorescence Resonance Energy Transfer (FRET) to quickly identify interactions. The underlying theory of the techniques is that when two molecules are close in space, i.e, interacting at a level beyond background, a signal is produced or a signal can be quenched. Then, a variety of experiments can be performed, including, for example, adding in a putative activator. If the activator competes with the interaction between the two signaling molecules, the signals will be removed from each other in space, and this will cause a decrease or an increase in the signal, depending on the type of signal used. This decrease or increasing signal can be correlated to the presence or absence of the putative activator. Any signaling means can be used. For example, disclosed are methods of identifying a PKC-δ activator, comprising contacting a first molecule and a second molecule together in the presence of a putative activator, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative activator and the in absence of the putative activator, wherein a decrease in FRET in the presence of the putative activator as compared to FRET measurement in its absence indicates the putative activator enhances PKC-δ induced apoptosis. This type of method can be performed with a cell system as well.

Combinatorial chemistry includes but is not limited to all methods for isolating small molecules or macromolecules that are capable of interacting with a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A.,et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example an extracellular portion of PKC-δ is attached to a DNA binding domain of a transcriptional activation protein, such as Gal 4. By performing the Two-hybrid technique on this type of system, molecules that bind the extracellular portion of PKC-δ can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and 5,506,337), sulfamides (U.S. Pat. No. 5,618,825), and benzodiazepines (U.S. Pat. No. 5,288,514).

Screening molecules similar to known activators, such as AD198, for activation of PKC-δ is a method of isolating desired compounds.

As used herein combinatorial methods and libraries included traditional screening methods and libraries as well as methods and libraries used in interative processes.

ii. Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as PKC-δ, are also disclosed. Thus, the products produced using the molecular modeling approaches that involve the disclosed compositions, such as, AD198, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

### E. KITS

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits can include a proteasome inhibitor and a PKC-δ activator. For example, disclosed is a kit for treating multiple myelomas, comprising the kit described above.

F. Compositions with Similar Functions

It is understood that the compositions disclosed herein have certain functions, such as activating PKC-δ. Disclosed herein are certain structural requirements for performing the disclosed functions, and it is understood that there are a variety of structures which can perform the same function which are related to the disclosed structures, and that these structures will ultimately achieve the same result, for example stimulation or activation of PKC-δ.

Also disclosed are other compounds related to a proteasome inhibitor that can be used interchangeably throughout relating to the methods of treatment disclosed herein. For example, a proteasome inhibitor belongs to the dipeptidyl boronic acids, which are proteasome inhibitors. For example, Adams et al. (Bioorg Med Chem Lett. 1998 Feb 17;8(4):333-8, herein incorporated in its entirety for reference to dipeptidyl boronic acids) describes potent and selective dipeptidyl boronic acid proteasome inhibitors.

### G. METHODS 0F MAKING PKC-δ ACTIVATORS

The compositions disclosed herein and the compositions necessary to perform the disclosed methods can be made using any method known to those of skill in the art for that particular reagent or compound unless otherwise specifically noted.

Disclosed are processes for making the compositions as well as making the intermediates leading to the compositions. For example, disclosed are PKC-δ activators. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO: 2 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO: 2 and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO: 2 and a sequence controlling the expression of the nucleic acid.

Disclosed are nucleic acid molecules produced by the process comprising linking in an operative way a nucleic acid molecule comprising a sequence encoding a peptide and a sequence controlling an expression of the nucleic acid molecule.

Disclosed are nucleic acid molecules produced by the process comprising linking in an operative way a nucleic acid molecule comprising a sequence encoding a peptide having 80% identity to a peptide and a sequence controlling an expression of the nucleic acid molecule.

Disclosed are nucleic acids produced by the process comprising linking in an operative way a nucleic acid molecule comprising a sequence encoding a peptide having 80% identity to a peptide, wherein any change are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals used in the methods of screening disclosed herein. Also disclose are animals produced by the process of adding to the animal any of the cells disclosed herein.

### H. VECTORS

Also disclosed are vectors comprising a nucleic acid encoding PKC-δ, and methods of administering to a subject a vector comprising a nucleic acid encoding PKC-δ. Such methods are useful in combination therapy with a proteasome inhibitor. It has been shown that PKC-δ is lethal to normal and neoplastic keratinocytes when overexpressed by an adenoviral vector and activated by TPA. Lethality is rapid, does not require new protein synthesis, and is prevented by selective inhibitors of PKC-δcatalytic activity, showing that this kinase targets substrates that are involved in a death pathway. PKC-δ adenoviral vectors were able to produce high levels of gene expression in human keratinocytes and caused growth inhibition and transglutaminase I induction. Furthermore, PKC-δ caused a spindle cell morphological change. It has been shown that a lethal response is a general effect of activation of the PKC-δ isoform when overexpressed in squamous cell types.

Vectors comprising a nucleic acid encoding PKC-δ are useful in cell assays, as well as in transgenic animals. These vectors are also useful in gene therapy applications to treat diseases and mediate apoptosis.

In the methods described herein which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

1. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modifed to specifically function with large DNA molecules. Further, these methods can be used to target certain diseases and cell populations by using the targeting characteristics of the carrier.

i. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as that encoding PKC-δ (SEQ ID NO: 2) into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

ii. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

iii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

iv. Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

v. Large payload viral vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson,. Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

2. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes 25 place, causing the delivered nucleic acid to be integrated into the host genome. These systems and the methods necessary to promote homologous recombination are known to those of skill in the art.

In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

i. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

ii. Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J Molec. Appl, Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

The present invention is more particularly described in the following examples which are intended as illustrative only since numerous modifications and variations therein will be apparent to those skilled in the art.

### I. EXAMPLES

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### 1. Example 1

### Mitochondrial Damage Induced by Bortezomib is Mediated by Accumulation of Active Protein Kinase C-δ in Mitochondria

i. Materials and Methods

U937 cells were grown in RPMI with 10% fetal bovine serum and L-glutamine. Bortezomib was from Millennium Pharmaceutical Corp. (Cambridge, Mass.). Rottlerin was from Sigma (St. Louis, Mo.). PKC-δ monoclonal antibody was from BD Transduction Laboratories (San Jose, Calif.). Boc-D(Ome)-FMK (BAF) and Go6976 were from Calbiochem (San Diego, Calif.). The TUNEL assay (terminal deoxynucleotidyltransferase-mediated dUTP-biotin nick end labeling) was from Roche (Switzerland). Subcellular fractionation and western blotting were performed as described (Chen Jet al. Mol Cancer Ther. 2002;1:961-967).

ii. The Role of PKC-δ in Bortezomib-Induced Apoptosis

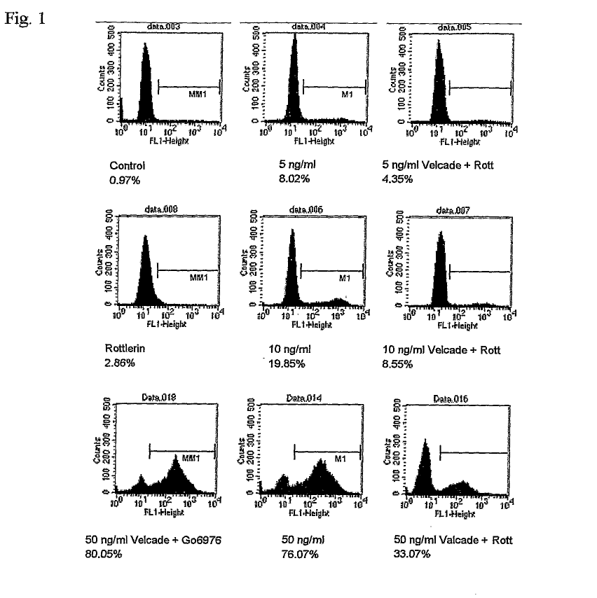

The sensitivity of U937 cells to bortezomib was examined. Cells became TUNEL positive in 5 ng/ml bortezomib, and IC₅₀ was between 10 to 50 ng/ml. A time course analysis using 50 ng/ml of bortezomib was then performed. Cells did not become TUNEL positive at 6 hours, but became strongly positive after 16 h of incubation (FIG. 1). The role of PKC-δ in bortezorib-induced apoptosis was then studied using a PKC-δ inhibitor, rottlerin. At concentrations of 5, 10 and 50 ng/ml, bortezomib induced 8.02%, 19.85% and 76.07% respectively of apoptosis by TUNEL assays after 16 hours of incubation. The percentages of TUNEL positive cells decreased dramatically to 4.35%, 8.55% and 33.07%, respectively, with addition of rottlerin. In contrast, Go6976, a potent inhibitor of classic PKCs (α, δ and γ), could not prevent bortezomib-induced apoptosis. This finding confirmed that PKC-δ plays an important role in bortezomib-induced apoptosis (FIG. 1).

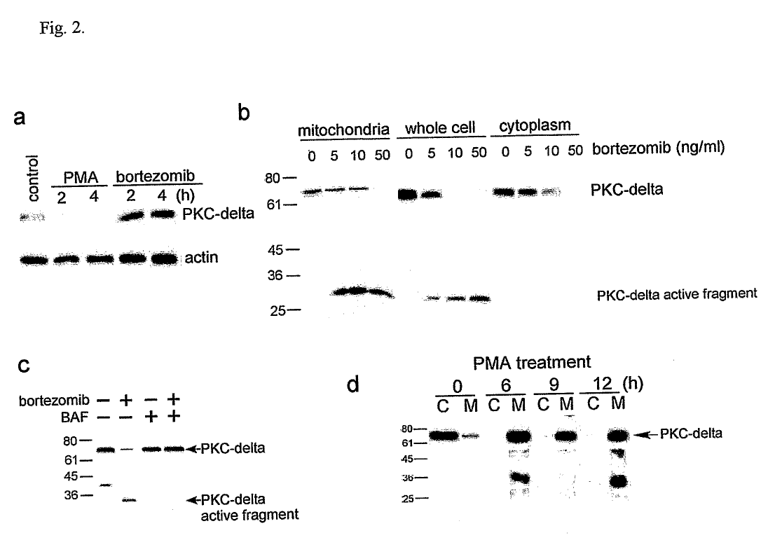

Next, PKC-δ protein was analyzed in U937 cells treated with bortezomib for 2 and 4 hours. Bortezomib induced an increase of PKC-δ, whereas phorbol ester PMA down-regulated PKC-δ at these two time points (FIG. 2a). When cells were incubated with bortezomib for 16 hours, the level of PKC-δ decreased, but there was a new band at 32 kD, which was recognized by the PKC-δ antibody (FIG. 2b). The intensity of the 32 kD band correlated directly with the doses of bortezomib. When the localization of this band was studied by subcellular fractionation, it was only present in mitochondria but not in the cytoplasm, where full-length PKC-δ is. This band is a fragment containing only the catalytic domain of PKC-δ that is generated by caspase cleavage as reported by Denning et al.

To prove that this fragment was indeed generated by caspase cleavage of PKC-δ, the cells were treated with bortezomib and a pan-caspase inhibitor BAF. The disappearance of this 32 kD band after BAF treatment confirmed that generation of this band can depend on caspases (FIG. 2c). This finding is consistent with two known mechanisms of PKC-δ activation. One is by phorbol ester, which releases the inhibition of the regulatory domain of PKC-δ from the catalytic domain. The other is by cleavage of the regulatory domain by caspase. The activated PKC-δ translocates to mitochondria. PMA was used to activate PKC-δ in U937 cells and detected full-length PKC-δ and its degradation products in mitochondria as reported (Majumder et al. Cell Growth Differ. 2001;12:465-470). In contrast, untreated control cells had all the PKC-δ present in the cytoplasm and no degradation products were detected (FIG. 2d).

This shows a mechanistic basis for the fact that myeloma cells primed with bortezomib have sensitized mitochondria to Bcl-2-targeting molecule HA14-1. Sequential treatment of myeloma cells with bortezomib followed by HA14-1 achieved the maximal effect rather than simultaneous or reverse order treatment. Hence there is a synergistic effect between bortezomib and mitochondria-targeting agents as a useful combination.

### 2. Example 2

### Protein Kinase C-δ and its Downstream Effectors as Potential Targets for Cancer Therapy

i. Biological Functions of the Protein Kinase C Family

Members of the Protein kinase C (PKC) family play diverse roles in many biological processes (Parker, 1997). Isoforms of the PKC family can be divided into three groups based on their interaction with and dependence on calcium and diacylglycerol. The classic PKCs, including isoforms α, βI, βII and γ, require both calcium and diacylglycerol for activation. The novel PKCs, including δ, ε, η, and θ, are independent of calcium but require diacylglycerol for activation. The atypical PKCs, including λ and ζ, are independent of both calcium and diacylglycerol. Each isoform plays different roles in cell growth, proliferation, differentiation or apoptosis (Ohno, 1997). Due to their important roles in many different cancers, PKCs can be used in cancer therapy.

ii. Protein Kinase C Isoforms in Apoptosis

The balance of survival and apoptotic signals determines cancer cell death, the ultimate goal of cancer therapy (Cory and Adams, 1998; Reed, 1999; Adams and Cory, 2001; Green and Evan, 2002). Induction of apoptosis through modulation of key factors in the apoptotic pathway can have a direct and dominant effect on cells. For example, the PKC isoform best characterized in triggering apoptosis is PKC-δ (Brodie and Blumberg, 2003). Compared with PKC-α, which provides a survival and proliferation signal, PKC-δ provides a direct target to enhance apoptosis (Mandil et al, 2001).

Involvement of PKC-δ in apoptosis was first demonstrated by activation of PKC-δ in cells treated with a variety of apoptotic stimuli, including H₂O₂, TNF-δ, the Fas ligand, UV and γ irradiation, and etoposide treatment. Inhibition of PKC-δ activity by a PKC-δ-specific inhibitor, rottlerin, or by a dominant negative mutant resulted in suppression of the apoptotic response. Furthermore, PKC-δ-deficient mice had an increased B cell population and formed numerous germinal centers in the absence of stimulation. The observed abnormal B cell proliferation was associated with enhanced autoimmunity due to the persistence of self antigen-recognizing B cells that failed to undergo apoptosis during positive selection. Analysis of these knockout mice thus established a role for PKC-δ in controlling B-cell apoptosis in the regulation of B cell tolerance.

iii. Regulation of the Activity of PKC-δ

Regulation of PKC-δ activity is mediated by at least three mechanisms. The regulatory C1 domain has an inhibitory effect on the catalytic domain found at the carboxyl terminus (Ohno, 1997; Parker, 1997). One way to release inhibition is by interaction of diacylglycerol or phorbol esters with the C1 domain, which triggers a conformational change. A second mechanism is mediated by cleavage of the catalytic domain from the C1 regulatory domain, which is achieved during apoptosis by activated caspase 3. Tyrosine phosphorylation of PKC-δ at Tyr64 and 187 is essential for the cleavage and the apoptotic effect of PKC-δ. Tyr311 phosphorylation by Lck kinase after H₂O₂ treatment enhances basal PKC-δ activity and elevates its maximal activity in the presence of diacylglycerol. Finally, activated PKC-δ undergoes ubiquitination and degradation through the proteasome pathway, which prevents a persistent effect of PKC-δ.

iv. Translocation of PKC-δ During Apoptotic Responses

Depending on the cell types and the apoptotic stimuli, PKC-δ has been reported to translocate to nearly all subcellular organelles, including nuclei, mitochondria, the Golgi complex, endoplasmic reticulum (ER) and the plasma membrane (Brodie and Blumberg, 2003; Roychowdhury and Lahn, 2003). At each subcellular organelle, PKC-δ phosphorylates different substrates, inducing various responses that eventually lead to cell death. Three criteria can be used for identification of a physiologic substrate of PKC-δ: (1) evidence that PKC-δ phosphorylates the protein, (2) evidence for their interaction, and (3) deletion or inactivation of the substrate leads to at least a partial loss of PKC-δ-induced response.

v. PKC-δ Substrates in the Nucleus

Translocation of PKC-δ to the nucleus has been established in T cells and C6 glioma cells. A putative nuclear localization signal has been identified at the carboxyl terminus of the catalytic domain of PKC-δ. Previously, nucleolin, which is required for nerve-growth factor (NGF)-induced differentiation of pheochromocytoma cells PC12, was identified as a substrate of PKC-ζ (Zhou et al, 1997). However, neither PKC-ζ nor PKC-δ can phosphorylate nucleolin, and nucleolin is not involved in the apoptotic response.

PKC-δ is responsible for constitutive and DNA damage-induced phosphorylation of Rad9, a key factor involved in checkpoint regulation of the DNA damage response (al-Khodairy et al, 1994). An interaction between PKC-δ and Rad9 was also demonstrated, and showed that PKC-δ phosphorylated Rad9 both in vitro and in cells treated with Cytarabine (ara-C) or γ-irradiation. Nuclear Rad9 forms a critical heterotrimeric complex with Hus1 and Rad1, the 9-1-1 complex that is involved in DNA damage checkpoint control. PKC-δ which translocated to the nucleus during apoptosis, enhanced the phosphorylation of Rad9 and the formation of the Rad9-Hus1-Rad1 complex. Interestingly, Rad9 is also phosphorylated by ATM and c-Ab1. The latter kinase also interacts with PKC-δ. Using ATM siRNA to down-regulate the level of ATM, a diminished nuclear targeting of PKC-δ was observed, showing that ATM is required for nuclear targeting of PKC-δ and is functionally upstream of PKC-δ. This shows a direct linkage between PKC-δ and DNA damage-induced checkpoint regulation, elucidating the mechanism of PKC-δ-induced apoptosis.

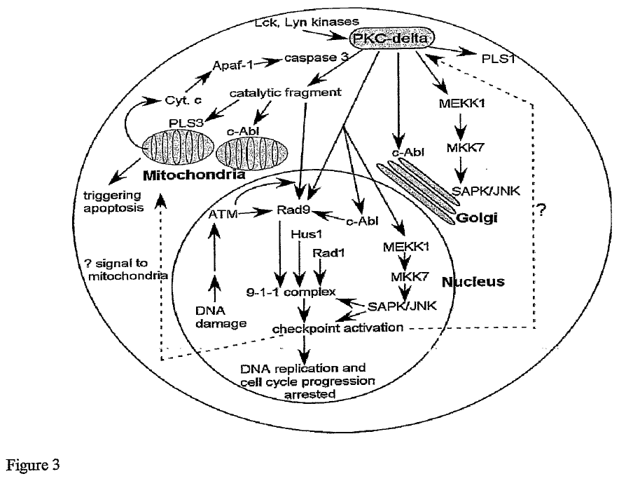

Another downstream effector of PKC-δ in DNA damage response in cells treated with ara-C is stress-activator protein kinase (SAPK/JNK) (FIG. 3). DNA damage-induced SAPK/JNK activation was attenuated by rottlerin, a dominant negative mutant of PKC-δ and PKC-δ siRNA. PKC-δ did not directly phosphorylate SAPK/JNK, rather SAPK/JNK was indirectly phosphorylated through the mitogen-activated protein kinase (MAPK) pathway, PKC-δ→MEKK1→MKK7→SAPK/JNK. The finding that SAPK/JNK is a downstream effector of PKC-δ provides another mechanism of PKC-δ-induced apoptosis. Interestingly, SAPK/JNK was shown to be the substrate of PKC-β and to translocate to mitochondria after phosphorylation to induce cytochrome c release (Ito et al, 2001a).

vi. PKC-δ Substrates in the Mitochondria

Translocation of PKC-δ to mitochondria was shown in U937 myeloid leukemia cells and keratinocytes. The translocation can be induced by phorbol ester and oxidative stress. With UV irradiation, mitochondrial targeted PKC-δ was cleaved by caspase 3 to generate the active catalytic fragment of PKC-δ. One substrate of PKC-δ is c-Ab1 kinase. It has been demonstrated that PKC-δ interacts with c-Ab1, and that the phosphorylation of c-Ab1 results in activation of c-Ab1 kinase. Cells treated with H₂O₂ had an increase in c-Ab1 activity, which was attenuated by the PKC-δinhibitor, rottlerin, and by overexpression of the regulatory domain of PKC-δ. In the unstimulated condition, c-Ab1 localized to the nucleus, ER and cytoplasm. On ER stress caused by calcium ionophore A23187, brefeldin A or tunicamycin treatment, c-Ab1 translocated to mitochondria.

The second mitochondrial target of PKC-δ is the phospholipid scramblase 3 (PLS3), a member of the scramblase family that is responsible for bidirectional movement of phospholipids in the lipid bilayer. Unlike PLS1, which is localized in the plasma membrane (Zhou et al, 1997; Sims and Wiedmer, 2001), PLS3 is found exclusively in the mitochondria (Liu et al, 2003a, b). PLS3 is likely involved in translocation of cardiolipin from the mitochondrial inner membrane to the outer membrane during apoptosis. Mitochondria with expression of an inactive mutant of PLS3 have a low level of cardiolipin and poor respiration (Liu et al, 2003b). They also display a unique morphology, being larger in size, fewer in number, and with tightly packed cristae, consistent with the notion that PLS3 moves phospholipids from the inner membrane to the outer membrane (Liu et al, 2003b).

Cardiolipin translocation is directly tied to the sensitivity of mitochondria to tBid-induced cytochrome c release. Because tBid targeting to the mitochondria is mediated by cardiolipin (Lutter et al, 2000), the translocation of cardiolipin from the inner membrane to outer membrane facilitates the recruitment of tBid (Liu et al, 2003b). This idea was confirmed by the finding that mitochondria overexpressing PLS3 were more sensitive to tBid-induced cytochrome c release, whereas those expressing inactive mutant PLS3 were more resistant (Liu et al, 2003b).

PLS3 fulfills the three criteria we defined for a physiological substrate of PKC-δ. PLS3 can interact with PKC-δ and be phosphorylated by PKC-δ in vitro. HeLa cells expressing PLS3 become more positive in TUNEL studies when they were treated with the phorbol ester, PMA. Expression of mitochondria-targeted PKC-δ in cells resulted in apoptosis, and overexpression of PLS3 enhanced this effect. In contrast, overexpression of the inactive PLS3 mutant did not generate this response. These data support the view that PLS3 is a mitochondrial target of PKC-δ-induced apoptosis.

vii. PKC-δ Substrates in the Plasma

Another member of PLS family, PLS1, has been shown to be a target of PKC-δ in the plasma membrane (Frasch et al, 2000). PLS1 is phosphorylated by PKC-δ at a PKC phosphorylation consensus site, Thr161. Co-expression of PKC-v and PLS1 significantly increased the activity of scramblase following PMA treatment (Frasch et al, 2000). In contrast, co-expression of PKC-δ and a T161A mutant of PLS1 showed no increase in scramblase activity, indicating that phosphorylation of Thr161 by PKC-δ is important for scramblase function. In addition, PLS1 can be phosphorylated by c-Ab1, a kinase known to interact with PKC-δ in other organelles.

During apoptosis, phosphatidylserine (PS) translocates from the inner leaflet to the outer leaflet of the plasma membrane. The regulation of transbilayer movement of phospholipids is controlled by at least three enzymes. One is aminophospholipid translocase, or flippase, which moves phospholipids inwards. One is the phospholipid scramblase (PLS1) that moves phospholipids bidirectionally. The third is a less well-characterized outward-directed floppase (Bevers et al, 1999). Probably due to the complexity of the regulation of phospholipid topology in lipid bilayers, cells from mice which are homozygous for a deletion of PLS1 still maintain their ability to translocate PS to the surface (Zhou et al, 2002). This can be due to compensation by aminophospholipid translocase activity.

A second plasma membrane target of PKC-δ is Fyn kinase, found in the plasma membrane of platelets. In platelets, activation of PKC by phorbol ester induces platelet degranulation and activation of the integrin α_{IIb}β_{III}. It has been demonstrated that activation of the platelet adhesion complex is associated with interaction of Fyn kinase and PKC-δ, but not other members of the PKC family. Fyn kinase is also phosphorylated at a serine residue that is found within a PKC consensus sequence.

viii. PKC-δ is a Survival Factor in Several Cancer Cells Using PKC-δ as a Therapeutic Target

Based on the ample evidence that PKC-δ enhances apoptotic responses in certain systems, PKC-δa can be targeted in cancers in which PKC-δ is known to play a pro-apoptotic, but not a pro-survival, role. An example of a compound that can accomplish this is a derivative of adriamycin, N-benzyladriamycin-14-valerate (AD198), which does not inhibit topoisomerase II and binds DNA weakly in contrast to its parental drug adriamycin. AD198 has a potent anti-tumor effect through activation of PKC-δ by interacting with the regulatory domain of PKC-δ like the phorbol ester. More importantly, AD198 can override the anti-apoptotic effect of Bcl-2, a common problem in many malignancies.

Another approach is gene therapy to introduce PKC-δ by adenoviral vectors, which have been achieved in cell lines. Other approaches include blocking PKC-δ degradation through inhibition of the ubiquitin-proteasome pathway, for which bortezomib (PS-341) is available. It has been observed that bortezomib induces activation and accumulation of PKC-δ in mitochondria and that the PKC-δ inhibitor rottlerin compromises the apoptotic effect of bortezomib. Finally, modulation of tyrosine phosphorylation of PKC-δ through blocking of Src kinase family and activation of PKC-δ is also possible.

ix. Using Downstream Effectors of PKC-δ as Therapeutic Targets

Given the dual roles of PKC-δ in promoting survival and apoptosis, downstream effectors of PKC-δ can be utilized as targets for induction of apoptosis in cancer therapy. One target in both the nucleus and mitochondria is c-Ab1, (STI-571, Gleevec), that blocks the tyrosine kinase activity of the fusion protein Bcr-Ab1, and effectively controls the proliferation and induces apoptosis in chronic myelocytic leukemia (CML) cells with minimal side effects. STI-571 is widely used to treat CML. With DNA damage-induced apoptosis, the kinase activity of c-Ab1 is activated by PKC-δ induced phosphorylation, and is required for the apoptotic response.

Overexpression of Rad9 in cells was shown to be pro-apoptotic; whereas down-regulation of Rad9 by an antisense vector suppressed apoptosis. It has been shown that Rad9 contains a BH3 domain and can interact with B c1-2 and Bcl-xL. Hence Rad9-induced apoptosis can be mediated by inhibition of Bcl-2 through its BH3 domain, or indirectly though the general DNA damage-induced apoptotic pathway.

Another target is PLS3, a member of the scramblase family that is present in the mitochondria. Its function is critical for mitochondria. It has been reported that overexpression of wild-type PLS3 enhanced UV-induced apoptosis; whereas expression of an inactive PLS3 mutant suppressed apoptosis. Using ³²P labeling and TLC analysis of the phospholipids in mitochondrial inner and outer membranes, it was found that PLS3 can be responsible for moving cardiolipin from the mitochondrial inner membrane to the outer membrane during apoptosis. The translocation of cardiolipin to the mitochondrial outer membrane enhanced sensitivity to tBid-induced cytochrome c release.

The MAPK pathway can also be targeted based on the observation that PKC-δ activates SAPK/JNK. The activation of the DNA-damage checkpoint is a protective mechanism to prevent cell cycle progression in the presence of DNA damage. When DNA damage becomes so severe that it is beyond the capacity of repair, the apoptotic pathway is activated. Inhibitors of various MAPK pathways are known in the art, and are able to block survival signals and down-regulate checkpoint regulation to facilitate the activation of apoptosis.

Those skilled in the art will recognize, or be able to ascertain using no more than routine experimentation, many equivalents to the specific embodiments of the method and compositions described herein. Such equivalents are intended to be encompassed by the following claims.

### 3. Example 3

### N-benzyl adriamycin-14-valerate (AD198) Induces Apoptosis Through Protein Kinase C-δ-Induced Phosphorylation of Phospholipid Scramblase 3

i. Materials and Methods

Materials—The cDNA encoding full-length PLS3 was cloned into the QIAexpress pQE30 vector (Qiagen, Valencia, Calif.) to tag PLS3 with 6 consecutive histidine residues (6×His tag) at N-terminus (pQE-PLS3). A point mutant PLS3(T21A) was generated using the Quick Change site-directed mutagenesis kit (Stratagene, La Jolla, Calif.). As there were two serine residues generated at the cloning region of pQE30, one before and one after the consecutive histidine residues, these two serines were mutated to alanine to abrogate possible false phosphorylation. Mammalian expression vectors for His-tagged PLS3 and PLS3(T21A) were construed with pCMV vector. Mammalian expression vectors for wild-type (pHA-PKC-δ) and kinase-defective PKC-δ (pHA-PKC-δ KD, K376R) were kindly provided by Dr. Jae-Won Soh (Herbert Irving Comprehensive Cancer Center, Columbia University, NY). Mammalian PKC-δ siRNA expression plasmid (pKD-PKC-δ-v3) was from Upstate (Lake Placid, N.Y.). The polyclonal antibody to PLS3 was raised in rabbits against full-length recombinant PLS3 (Proteintech Group Inc., Chicago, Ill.). The first 50-aa fragment of PLS3 was made as His-tagged protein similar to full-length PLS3. The monoclonal antibody against PKC-δ was obtained from BD Biosciences (Palo Alto, Calif.). Monoclonal antibodies to phosphothreonine (PT) and β-actin were obtained from Sigma-Aldrich, Inc. (St. Louis, Mo.). The polyclonal antibody to voltage-dependent anion channel (VDAC) was obtained from Affinity BioReagents (Golden, Colo.). Secondary anti-mouse or anti-rabbit antibodies conjugated with horseradish peroxidase, and protein G Sepharose beads were obtained from Amersham Pharmacia Biotech (Piscataway, N.J.). Recombinant human PKC-δ enzyme was purchased from Calbiochem Biosciences (La Jolla, Calif.). [γ-³²P] ATP was from Life and Analytical Sciences (Boston, Mass.). siRNA against PLS3 and a random sequence siRNA were from Qiagen. Z-VAD was from ICN Pharmaceuticals, Inc. (Aurora, Ohio) and cyclosporine A (CsA) was from Sigma-Aldrich, Inc. (St. Louis, Mo.). MitoTracker Green was from Molecular Probes, Inc. (Eugene, Oreg.). AD198 was provided by Dr. Mervyn Israel (Department of Pharmacology, the University of TN Health Science Center).

Cell culture, transfection and treatment—HeLa cells were cultured in Dulbecco's modified Eagle's medium (DMEM) containing 10% fetal bovine serum, 2 mM L-glutamine and penicillin (100 units/ml)-streptomycin (100 μg/ml) at 37° C. in a humidified 5% CO₂ atmosphere. HeLa cells at 90% confluence were transfected with different mammalian expression vectors using Lipofectamin 2000 according to the manufacturer's protocol (Invitrogen Inc., Carlsbad, Calif.). At 24 h or 48 h after transfection, cells were treated with 1:100 dilution of AD198 that was dissolved in dimethyl sulfoxide (DMSO). For down-regulation of PLS3, HeLa cells at 50% confluence were transfected with siRNA against PLS3 or a scrambled control. At 48 h, cells were treated with AD198 for 16 h and harvested for flow cytometry. The whole cell lysate was extracted for analysis with Western blotting.

Preparation of recombinant PLS3 proteins—His-tagged PLS3 proteins was generated with E. coli strain M15 [pREP4] containing the pQE-PLS3 or PLS3(T21A) after induction with 1 mM isopropyl-β-thiogalactoside (IPTG). Bacteria were lysed in a buffer containing 100 mM NaH₂PO₄, 10 mM Tris-HCl, 8 M urea, pH 8.0. His-tagged proteins were purified on nickel-nitrilotriacetic acid affinity beads (Ni-beads) and washed extensively with the same buffer at pH 6.3. Bound proteins were eluted in the same buffer at pH 4.5 as described by the manufacturer (Qiagen). The purity of the protein was examined by gel electrophoresis, followed by Coomassie blue staining.

Pulldown of His-tagged proteins from transfected HeLa cells—Transfected HeLa cells were washed with ice-cold phosphate-buffered saline (PBS) and incubated in the lysis buffer (50 mM NaH₂PO₄, 500 mM NaCl, 20 mM imidazole, 1% Triton X-100, 20 mM 2-mercaptoethanol, pH8.0) for 5 min and sonicated briefly on ice. The lysates were centrifuged at 10,000×g for 10 min at 4° C. The supernatant was saved and the protein concentration was measured with the Bio-Rad protein assay (Bio-Rad Laboratories, Hercules, Calif.) and adjusted to same concentrations for each sample. The supernatants were incubated with Ni-beads on a shaker for 2 h at 4° C. Beads were then pelleted at 800×g for 2 min at 4° C. and washed with ice-cold wash buffer (50 mM NaH₂PO₄, 500 mM NaCl, 20 mM imidazole, 1% Triton X-100, pH8.0) for five times. Beads were resuspended in SDS sample buffer and analyzed by Western blotting.

Preparation of whole cell lysates and subcellular fractionation—HeLa cells were washed with ice-cold PBS, lysed with ice-cold lysis buffer (50 mM Tris-HCl, pH7.4, 100 mM NaCl, 1% Triton X-100, 1 mM EDTA, and 1 nM phenylmethylsulfonyl fluoride for 5 min and sonicated briefly on ice. Cell lysates were centrifuged at 10,000×g for 10 min at 4° C. The supernatants were saved as the whole cell lysates and used for further immunoprecipitation or added into the SDS sample buffer for Western blotting. Subcellular fractionation was performed as described (Liu et al. (2003)).

Western blot analysis—Equal amounts of protein were analyzed by 10% SDS-PAGE and electrotransferred to Immobilon-P membranes (Millipore Corporation, Bedford, Mass.). Monoclonal PKC-δ, PT, and β-actin and polyclonal VDAC antibodies were used at 1:1,000 and polyclonal PLS3 antibody was used at 1:2,000 for immunoblotting. Secondary anti-mouse or anti-rabbit antibodies conjugated with horseradish peroxidase were used at 1:2,000 and incubated with the membrane for 1 h at room temperature. After washing 3 times with TBS/T (1×TBS and 0.1% Tween-20), blots were developed with enhanced chemiluminescence (ECL) reagents (Pierce, Rockford, Ill.).

Immunoprecipitation—Whole cell lysates were incubated with 1.25 μg/ml PKC-δ antibody at 4° C. for 2 h and then protein G Sepharose beads were added for an additional 2 h. After washing extensively with RIPA buffer (20 mM Tris-HCl, pH8.0, 1% NP-40, 0.2% deoxycholate, 120 mM NaCl), the pellets were resuspended in SDS sample buffer and subjected to Western blotting.

Analysis of apoptosis and determination of the transmembrane potential in mitochondria—Cell death was quantified by propidium iodide (PI) staining, followed by flow cytometry (Becton-Dickinson). HeLa cells were fixed in 0.5 ml cold 70% ethanol at −20° C. overnight. Cell pellet were resuspended in 300 μl of PBS containing 0.025 mg/ml PI, 0.05% Triton X-100, 0.1 mg/ml RNase A and incubated at room temperature for 30 min. The DNA content was evaluated by FACScan. For mitochondrial potential analysis, HeLa cells were incubated with MitoTracker Green at 37° C. for 20 min. The cells were collected and washed with PBS and analyzed by FACScan.

In vitro phosphorylation assay—In vitro phosphorylation was performed in a total volume of 60 μl of reaction mixture. Recombinant protein was diluted to less than 0.2 M urea immediately before phosphorylation assays. The phosphorylation mixture contained 0.1 μg recombinant human PKC-δ enzyme, 20 mM HEPES buffer (pH 7.4), 4 mM MnCl₂, 4 mM MgCl₂, 50 μM ATP, 20 μCi of [γ-³²P] ATP, and 1μg of recombinant PLS3 protein. The reaction mixture was incubated at room temperature for 20 min and terminated by adding 20 μl of 4×SDS sample buffer. The phosphorylated products were separated by SDS-PAGE, electrotransferred to the Immobilon-P membrane, and exposed by autoradiography.

ii. Results

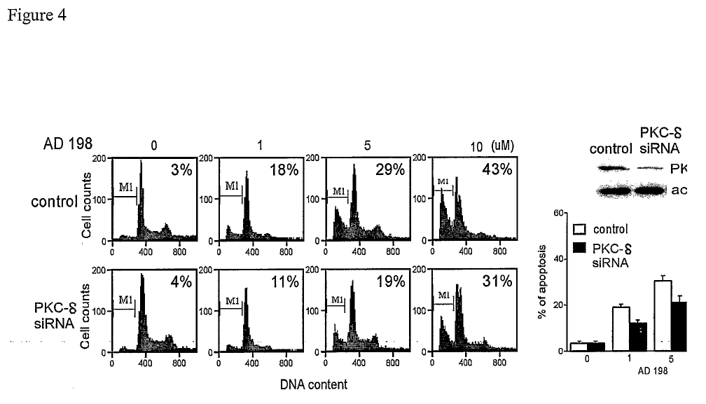

AD198-induced apoptosis is PKC-δ dependent—Based on the report that AD198 activated PKC-δ (Roaten et al. (2002), Minotti et al. (2004)), it was first examined whether AD198-induced apoptosis was suppressed by down-regulation of PKC-δ. HeLa cells were transfected with pKD-PKC-δ-v3 plasmid which expresses PKC-δ siRNA to down-regulate PKC-δ (FIG. 4). Cells were then treated with AD198 at 0, 1, 5 and 10 μM for 16 h. In all concentrations of AD198, less apoptosis was detected when PKC-δ was down regulated by siRNA (FIG. 4), confirming that AD198-induced HeLa cells apoptosis is PKC-δ dependent.

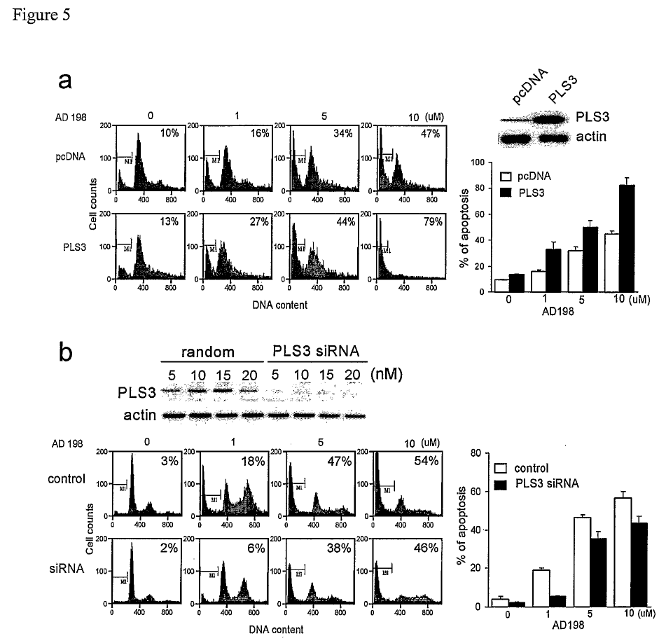

Overexpression of PLS3 enhances AD198-induced apoptosis—Since AD198-induced apoptosis is PKC-δ-dependent, it was evaluated whether PLS3, a mitochondrial substrate of PKC-6, was involved in AD198-induced apoptosis. HeLa cells were transfected with the vector to overexpress wild-type PLS3. Cells were then treated with AD198 at various concentrations for 16 h. At three different concentrations of AD198, HeLa cells with overexpression of PLS3 were more sensitive to AD198-induced apoptosis than those without PLS3 overexpression (FIG. 5a). These data indicate that overexpression of PLS3 sensitized HeLa cells to AD198-induced apoptosis.

siRNA was then used against PLS3 to down-regulate PLS3 (FIG. 5b). Flow cytometry demonstrated that AD198-induced apoptosis was suppressed in HeLa cells transfected with siRNA against PLS3 compared with HeLa cells transfected with scrambled siRNA control (FIG. 5b). This finding again supports that AD198-induced apoptosis is mediated by a PLS3-dependent pathway.

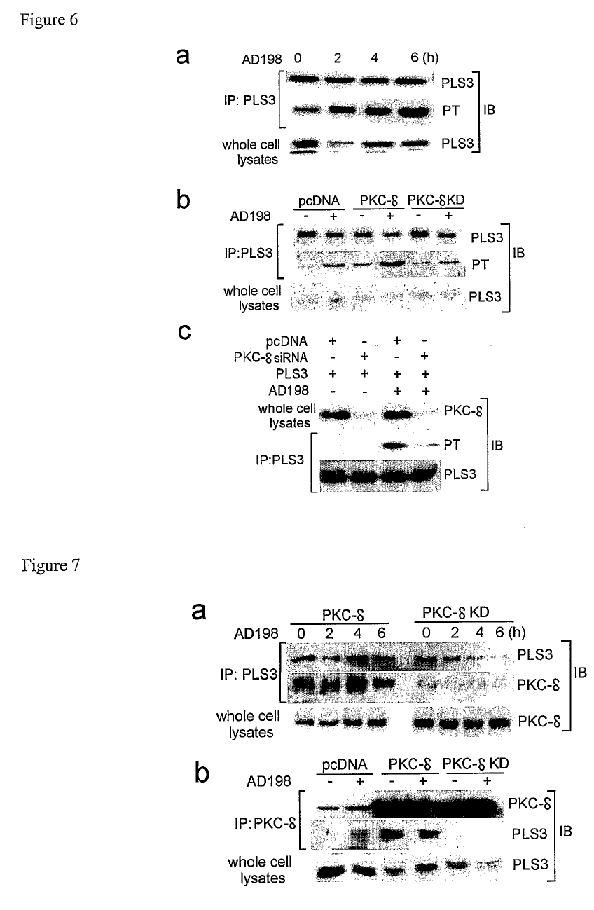

AD198 induces threonine phosphorylation of PLS3 by PKC-δ—It has been shown that UV treatment induces translocation of PKC-δ to mitochondria and phosphorylation of PLS3, and that phosphorylated PLS3 can be recognized by anti-PT antibody, but not by anti-phosphoserine antibody (Liu et al. (2003)). Herein, it was investigated whether AD198 can also induce PLS3 phosphorylation by activating PKC-δ. HeLa cells were transfected with the His-tagged PLS3 vector and treated with AD198 for 0, 2, 4, and 6 h. His-tagged PLS3 was pulled down with Ni-beads and phosphorylation of PLS3 was evaluated by Western blotting using PT antibody. In untreated cells, there was a baseline phosphorylation of PLS3 at threonine, and threonine phosphorylation steadily increased after AD198 treatment for at least 6 h (FIG. 6a). The same method of Ni-beads pull-down was then used to examine the effect of PKC-δ overexpression. We His-tagged PLS3 was co-transfected along with PKC-δ or kinase-defective PKC-δ KD into HeLa cells and then treated cells with AD198. Immonoblotting of His-tagged PLS3 with the PT antibody confirmed that AD198 treatment enhanced PLS3 phosphorylation at threonine, and that overexpression of PKC-δ further enhanced this process but the kinase-defective PKC-δ KD did not (FIG. 6b). The presence of PLS3 phosphorylation in cells transfected with kinase-defective PKC-δ indicated that the endogenous PKC-δ was not completely suppressed. When PKC-δ was down-regulated by siRNA, phosphorylation of PLS3 at threonine after AD198 treatment was also suppressed (FIG. 6c), indicating that PKC-δ is the kinase activated by AD198 to induce PLS3 phosphorylation. Examination of the interaction between PLS3 and PKC-δ after AD198 treatment showed that PKC-δ was pulled down with PLS3 in cells co-transfected with PKC-δ. However, the interaction between PLS3 and PKC-δ KD was much weaker and did not increase after AD198 treatment (FIG. 7a). Similar results were obtained by immunoprecipitation of PKC-δ. In control cells, the immunoprecipitate of endogenous PKC-δ contained PLS3, which increased after cells were treated with AD198. Overexpression of PKC-δ increased the amount of PLS3 in the immunoprecipitate of PKC-δ regardless of AD198 treatment (FIG. 7b). These studies, combined with the results from Ni beads pull-down, confirmed the association between PLS3 and PKC-δ. The interaction between PLS3 and PKC-δ occurs prior to PLS3 phosphorylation and phosphorylation can stabilize the interaction between PLS3 and PKC-δ. IP with PKC-δ antibody in lysates from HeLa-PLS3 cells expressing kinase-defective PKC-δ revealed that no interaction between PLS3 and kinase-defective PKC-δ (FIG. 7b, last two lanes). In this IP, endogenous PKC-δ was far less abundant than the kinase-defective PKC-δ, which explains the complete lack of PLS3 in this IP.

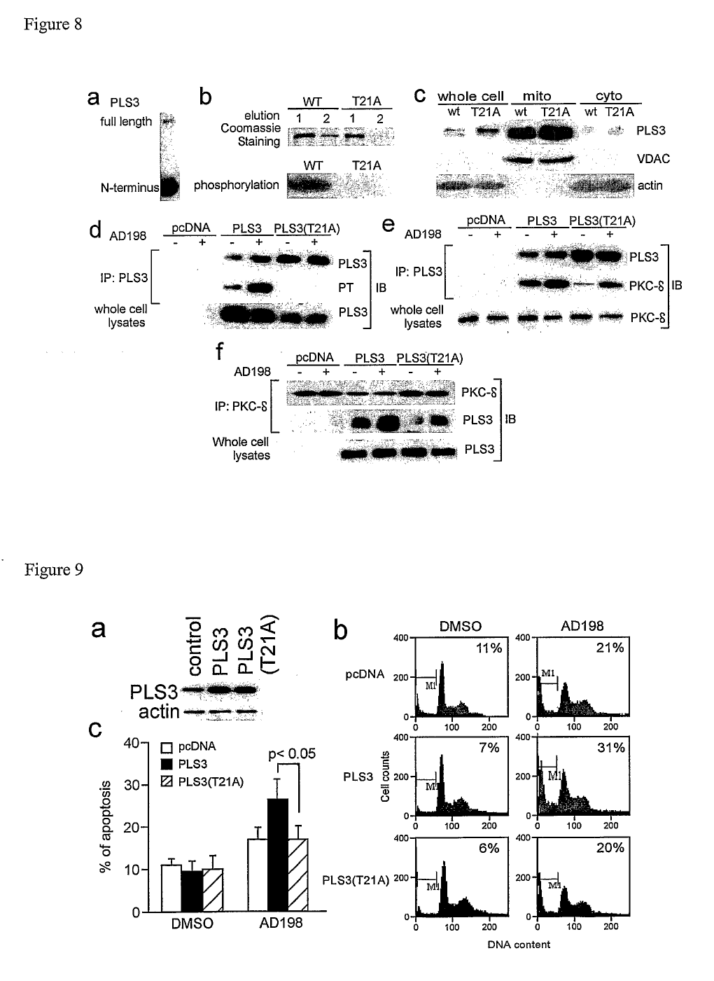

PKC-δ phosphorylates PLS3 at threonine 21—Since PLS3 is a high affinity substrate of PKC-δ (Liu et al. (2003)), the site of phosphorylation in PLS3 was investigated. In vitro phosphorylation of full-length and the 50-aa N-terminal fragment of PLS3 was performed. Phosphorylation was seen in both full-length PLS3 and its N-terminal fragment of PLS3 (FIG. 8a), which allowed a point mutation to be used to map the phosphorylation sites. Examination of the sequence of the first 50 amino acids of PLS3 revealed only a single threonine as the candidate phosphorylation site. Thus, Thr21 was mutated to alanine by site-directed mutagenesis, and generated recombinant PLS3(T21A) protein similar to wild-type PLS3 (FIG. 8b). In vitro phosphorylation revealed that mutation at Thr21 nearly eliminated PLS3 phosphorylation by PKC-δ (FIG. 8b), indicating that Thr21 is the site of phosphorylation in PLS3 by PKC-δ in vitro.

With the establishment of Thr21 as the site of PLS3 phosphorylation by PKC-δ in vitro, the mammalian expression vector pCMV-6His-PLS3(T21A) was transfected into HeLa cells to investigate the effect of T21A mutation in vivo. Subcellular fractionation of cells transfected with wild-type PLS3 or PLS3(T21A) revealed that mutation of this threonine residue did not affect mitochondrial targeting of PLS3 (FIG. 8c). After pulling down His-tagged PLS3(T21A) by Ni-beads, the PLS3(T21A) mutant could not be recognized by PT antibody even after AD198 treatment (FIG. 8d). In contrast, wild-type PLS3 was recognized by the same PT antibody and the signal increased after AD198 treatment. This finding confirmed that Thr21 is the primary site of PLS3 phosphorylation induced by AD198. Next T21A mutation was studied to determine if it affected the interaction between PLS3 and PKC-δ by co-transfection with PKC-δ. As shown in FIG. 8e, the His-tagged PLS3 pulled down by Ni-beads contained PKC-δ, but PLS3(T21A) was less effective in binding PKC-δ compared to wild-type PLS3 before AD198 treatment even though the amount of PLS3(T21A) was higher by blotting the same blot with PLS3 antibody (last two lanes, FIG. 8e). Upon incubation with AD198, there was increased association of PLS3 and PKC-δ for wild-type PLS3 versus mutant PLS3(T21A). The reciprocal immunoprecitation with the PKC-δ antibody showed a similar finding. Cells treated with AD198 had more PLS3 present in the PKC-δ IP. The PLS3(T21A) mutant has a weaker interaction with PKC-δ (FIG. 8f). These results indicate that the interaction between PLS3 and PKC-δ was compromised in PLS3(T21A), and therefore AD198-induced association between PLS3 and PKC-δ requires phosphorylation of Thr21 in PLS3.

PLS3(T21A) does not enhance AD198-induced apoptosis—If PLS3 phosphorylation at Thr21 is critical for the apoptotic effect of AD198-activated PKC-δ, it was predicted that overexpression of PLS3(T21A) would not be able to enhance AD198-induced apoptosis similar to the wild-type PLS3 (FIG. 5a). To test this possibility, HeLa cells transfected with vectors were treated to express wild-type PLS3 or PLS3(T21A) with AD198, and apoptosis was analyzed by PI staining and flow cytometry. Wild-type PLS3 increased the percentage of AD198-induced apoptosis from 7% to 31%, whereas cells expressing PLS3(T21A) had the same degree of apoptosis as the control cells after AD198 treatment (FIG. 9). It was concluded that AD198-induced PKC-δ activation and phosphorylation of PLS3 at Thr21 are critical to AD198-induced apoptosis.

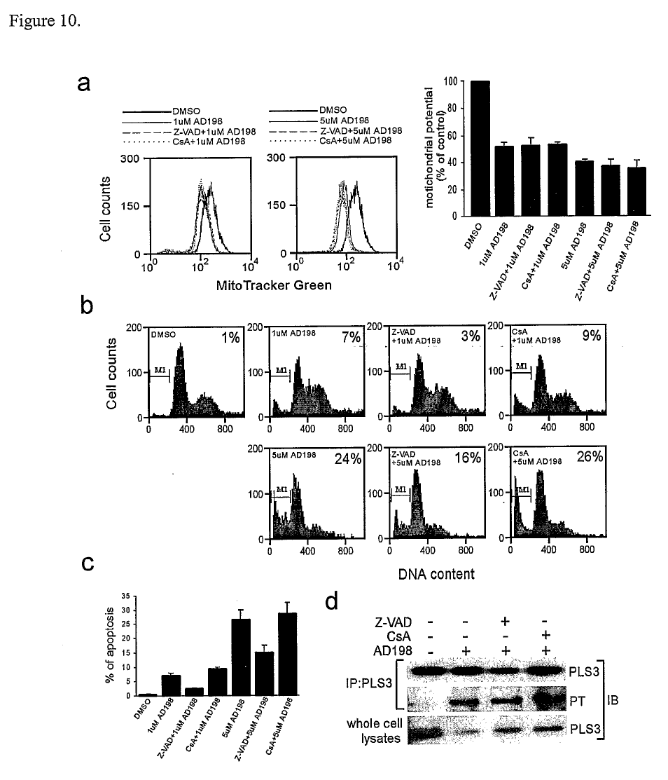

AD198-induced PLS3 phosphorylation is an upstream event of caspase activation and independent of mitochondrial permeability transition—Finally it was investigated how PLS3 phosphorylation was positioned in the apoptosis pathway. HeLa cells were treated with a pan-caspase inhibitor Z-VAD followed by AD198 treatment. Z-VAD could not block AD198-induced mitochondrial permeability transition but suppressed AD198-induced apoptosis (FIGS. 10a, b, c). It was also determined whether PLS3 was phosphorylated in the presence of Z-VAD by pulling down His-tagged PLS3 with Ni beads and probed with PT antibody. Threonine phosphorylation of PLS3 after AD198 treatment was not affected by Z-VAD (FIG. 10d), showing that AD198-induced PLS3 phosphorylation occurs at the upstream of caspase activation. Similar studies were also performed to examine whether AD198-induced PLS3 phosphorylation is up- or down-stream of mitochondrial permeability transition. When the mitochondrial permeability transition pore complex was blocked by CsA, AD198-induced loss of mitochondrial potential and PLS3 phosphorylation were not affected (FIGS. 10a, b, c, d), indicating that PLS3 phosphorylation occurs independent of mitochondrial permeability transition.

iii. Discussion

PKC-δ plays an important role in the process of cell death by translocating to different organelles to induce apoptosis. Identification of substrates in each organelle is essential for understanding the mechanism of PKC-δ-induced apoptosis. The critical substrates identified so far include Rad9 in the nucleus (Yoshida et al. (2003)) and phospholipid scramblase 1 (PLS1) in the plasma membrane (Frasch et al. (2000)). Identification of the physiologic substrates in mitochondria is especially important since mitochondria are the integrators of apoptosis. We have shown that PLS3 is a substrate of PKC-δ upon translocation to mitochondria (Liu et al. (2003)). In the current study, an extranuclear-targeted anthracycline derivative AD198 was utilized, which binds to the C1b regulatory domain of PKC similar to phorbol ester (Roaten et al. (2001)), to activate PKC-δ and to investigate the effect of PLS3 in PKC-δ-induced apoptosis. It was found that overexpression of PLS3 enhanced the sensitivity of cells to AD198-induced apoptosis. In contrast, down-regulation of PLS3 by siRNA resulted in decreased apoptosis by AD198. These findings confirmed that PLS3 is a downstream effector of PKC-δ in mitochondria when PKC-δ is activated by AD198 and translocates to mitochondria.

To investigate the mechanism of mitochondrial damage induced by PKC-δ phosphorylated PLS3, it was determined that AD198-activated PKC-δ induced phosphorylation of PLS3 at threonine. The phosphorylated threonine was mapped to residue 21. This residue was confirmed to be the phosphorylated threonine in vivo since mutation to alanine prevented its recognition by the PT antibody (FIG. 8d) despite the fact that the PLS3(T21A) mutant was still capable, though weaker, of interacting with PKC-δ (FIGS. 8e, f). Phosphorylation may not be required for the interaction between PLS3 and PKC-δ as interaction was observed before cells were treated with AD198 (FIGS. 7, 8e and 8f). Phosphorylation of PLS3 by PKC-δ may stabilize the interaction between the two proteins. Cells overexpressing the PLS3 (T21A) mutant, although still undergoing apoptosis after treatment with AD198, were comparable to cells expressing the empty vector. This observation was in contrast with the enhanced sensitivity to AD198 when cells were transfected with the vector to overexpress wild-type PLS3 (FIG. 9).

The PKC-δ-induced PLS3 phosphorylation is apparently an early event in AD198-induced apoptosis. The presence of caspase inhibitor Z-VAD suppresses AD198-induced apoptosis, but can not prevent PLS3 phosphorylation. Thus PKC-δ-induced PLS3 phosphorylation is an upstream event of caspase in the apoptotic pathway. When CsA was added with AD198, mitochondrial potential was still disrupted by AD198, and apoptosis occurred. AD198-induced PLS3 phosphorylation was not affected by CsA either. This finding indicated that PLS3 phosphorylation by PKC-δ is independent of mitochondrial permeability transition.

The major advantage of using AD198 to investigate PLS3-mediated mitochondrial damage is that AD198 induces translocation of the activated PKC-δ to mitochondria. This is in contrast to other apoptotic agents, such as H₂O₂, UV, γ-irradiation or other chemotherapeutic agents, which induce PKC-δ activation and translocation to other organelles in addition to mitochondria (Brodie and Blumberg (2003), Liu et al. (2003)). Nuclear targeted PKC-δ can phosphorylate Rad9, which is a critical mediator of the apoptotic process. Using an extranuclear-targeted anthracycline like AD198 will eliminate the contribution from nuclear-targeted PKC-δ. With the central role of mitochondria in apoptosis, the exclusive mitochondrial effect of AD198 is considered an advantage in directly inducing apoptosis. The fact that AD198 overcomes resistance due to Bcl-2 overexpression (Barrett et al. (2002), Bilyeu et al. (2004)) is particularly attractive since Bcl-2/Bcl-xL overexpression is a common mechanism of drug resistance in clinical application of chemotherapeutic agents. Further development of novel strategies to manipulate the PKC-δ/PLS3 pathway in mitochondria is a promising way to develop novel therapy for cancer.

### 4. Example 4

### Protein Kinase C-δ-Induced Apoptosis is Mediated by Phosphorylation and Activation of Phospholipid Scramblase 3

i. Materials and Methods

Materials—PLS3(T21A) and (T21D) mutant was generated using the Quick Change site-directed mutagenesis kit (Stratagene, La Jolla, Calif.). His-tagged PLS3 or mutants were cloned into the QIAexpress pQE30 vector (Qiagen, Valencia, Calif.) (pQE-PLS3) for generation of recombinant proteins as described. Mammalian expression vectors for His-tagged PLS3 and PLS3(T21D) were constructed with pcDNA3.1 vector respectively. The siRNA to down-regulate PLS3 and scrambled control were from Qiagen. The cDNAs encoding PLS3* and PLS3(T21D)* contain an additional single base mutation G99(GGC) to G99(GGA) at the center of PLS3-siRNA corresponding region so that the expression of the transgenes is not affected by PLS3 siRNA and can be used for rescuing. Mammalian PKC-δ siRNA expression plasmid (pKD-PKC-δ-v3) was from Upstate (Lake Placid, N.Y.). PLS3 polyclonal antibody was raised in rabbits against full-length recombinant PLS3 (Proteintech Group Inc., Chicago, Ill.). PKC-δ monoclonal antibody was obtained from BD Biosciences (Palo Alto, Calif.). β-actin monoclonal antibody was obtained from Sigma-Aldrich, Inc. (St. Louis, Mo.). The polyclonal antibody against voltage-dependent anion channel (VDAC) was obtained from Affinity BioReagents (Golden, Colo.). Secondary antibodies conjugated with horseradish peroxidase were obtained from Amersham Pharmacia Biotech (Piscataway, N.J.). AD198 was provided by Dr. Mervyn Israel (Department of Pharmacology, the University of Tennessee Health Science Center) (Harstrick et al. Anticancer Drugs 6:681-685 (1995)). Recombinant tBid(G94E) proteins was generated and purified as described (Liu et al. Apoptosis 9:533-541 (2004)) and labeled with fluorescein isothiocyanate (FITC), FITC-tBid(G94E), in a carbonate-bicarbonate (1000 μg protein: 45 μg FITC, pH 9.0) and purified through Sephadex G25 gel filtration column (Amersham Pharmacia Biotech, Piscataway, N.J.). 10-N-nonyl acridine orange (NAO) was purchased from Molecular Probes (Eugene, Oreg.). 1-lauroyl-2-(1′pyrenebutyroyl)-sn-glycero-3-phosphocholine (pyrene-PC) was purchased from Avanti Polar Lipids (Alabaster, Ala.).

Cell culture, transfection, selection and treatment—HeLa cells were cultured in Dulbecco's modified Eagle's medium (DMEM) with 10% fetal bovine serum, 2 mM L-glutamine, 100 units/ml penicillin and 100 μg/ml streptomycin at 37° C. HeLa cells at 90% confluence were transfected with different mammalian expression vectors or siRNA using Lipofectamin 2000 according to the manufacturer's protocol (Invitrogen Inc., Carlsbad, Calif.). At 48 h after transfection, cells were incubated with 5 μM AD198 or dimethyl sulfoxide (DMSO) for 4 h or 16 h. After transfection, cells were selected with 1 mg/ml G418 for 2 wk. After corresponding mock-transfected cells were killed by G418, the concentration of G418 was decreased to 0.5 mg/ml and maintained for another 2 wk. The whole cell lysates or subcellular fractions were extracted for Western blotting analysis (Liu et al. (2003)).

Generation of recombinant PLS3 proteins—His-tagged PLS3 proteins was produced with E. coli (M15 [pREP4]) transformed with pQE-PLS3. Bacteria were induced with 1 mM isopropyl-β-thiogalactoside (IPTG) for 4 h before harvest. Bacterial pellets were resuspended and stirred gentlemanly in 100 mM NaH₂PO₄, 10 mM Tris-HCl, 8 M urea, pH 8.0 for 1 h at room temperature. After centrifugation, the supernatants were mixed with nickel-nitrilotriacetic acid affinity beads (Ni-beads) to pull down the His-tagged protein. The beads were washed twice with 100 mM NaH₂PO₄, 10 mM Tris-HCl, 8 M urea, pH 6.3. Bound proteins were renatured using a linear 7 M-0.1 M urea gradient in 500 mM NaCl, 20% glycerol, 20 mM Tris-HCl, pH 7.4. The renatured proteins were then eluted with 50 mM NaH₂PO₄, 300 mM NaCl, 250 mM imidazole, pH 8.0 as described by the manufacturer (Qiagen) and dialyzed in 50 mM Tris, 0.2 mM EGTA, 1 mM MgCl₂, 120 mM NaCl, 0.5% Triton X-100, 5% Na cholate hydrate, pH 7.4.

Preparation of whole cell lysates and subcellular fractionation—HeLa cells were harvested with the lysis buffer (50 mM Tris-HCl, pH7.4, 100 mM NaCl, 1% Triton X-100, 1 mM EDTA, and 1 mM phenylmethylsulfonyl fluoride) on ice for 5 min and sonicated briefly. The lysates were centrifuged at 10,000×g for 10 min at 4° C. and the supernatants were saved as the whole cell lysates for Western blotting. Subcellular fractionation was performed as described (Liu et al. (2003)) and isolated mitochondria were used for tBid(G94E)-binding analysis.

Western blotting analysis—Samples were separated by 10% SDS-PAGE and electrotransferred to the Immobilon-P membrane (Millipore Corp., Bedford, Mass.). The membrane was blotted with 1:1,000 primary antibody overnight at 4° C. and 1:2,000 secondary antibody for 1 h at room temperature. Enhanced chemiluminescence (ECL) reagents (Pierce, Rockford, Ill.) were used to develop the blots.

Analysis of apoptosis—HeLa cells were harvested and fixed in 70% ethanol at −20° C. overnight. Cells were then washed with PBS and stained with 0.025 mg/ml propidium iodide (PI), 0.05% Triton X-100, 0.1 mg/ml RNase A for 30 min at room temperature. The DNA content was analyzed by FACScan (Becton-Dickinson), and the subG₀/G₁ population was used to represent apoptosis.

Analysis of the tBid-binding capacity of mitochondria—Isolated mitochondria were incubated with FITC-tBid(G94E) in 50 μl reaction buffer (250 mM sucrose, 20 mM HEPES, 10 mM KCl, 3 mM KH₂PO₄, 1.5 mM MgCl₂, 1 mM EGTA, 0.5% mg/ml BSA, PH 7.4) for 30 min at room temperature. Mitochondria were pelleted at 10,000×g and washed extensively with the reaction buffer to remove unbound FITC-tBid(G94E) and the fluorescence intensity was quantified with Bio-Tek microplate reader.

Analysis of CL distribution in the mitochondria outer membrane—NAO was used to determine the distribution of CL in mitochondria (Garcia Fernandez, et al. Anal Biochem 328:174-180 (2004)). When NAO binds CL at 2:1 ratio (in the saturated state), fluorescence emission occurs at 630 nm. When cells were incubated with increasing concentrations of NAO, NAO saturates CL in the outer member first and then the inner member of mitochondria. Lower concentrations of NAO (2.5-12 μM) were used to saturate CL in the mitochondrial outer member and to measure the amount of CL in that compartment. At 35 μM NAO, all CL in mitochondria will be saturated with NAO and the fluorescence intensity represents the total amount of CL in mitochondria (Liu et al. (2003), Garcia Fernandez, et al. (2004)). HeLa cells were fixed with 1% paraformaldehyde in phosphate buffered saline (PBS) for 10 min at room temperature. Cells were washed with PBS and incubated with different concentrations of NAO for 15 min at room temperature followed by flow cytometry. The red fluorescence intensity was measured and the median value at each concentration was used to calculate the percentage of total amount CL versus the maximal fluorescence at 35 μM NAO treated control cells.

Lipid flip-flop analysis—Recombinant Bid was produced as glutathione-S-transferase fusion protein in BL21 bacteria and purified by glutathione Sepharose 4B affinity beads. tBid was produced by cleavage with full-length Bid with recombinant caspase 8 (Calbiochem, San Diego, Calif.) before usage. Some of the initial transbilayer diffusion studies were carried out with tBid kindly provided to us by Jean-Claude Martinou (University of Geneva, Switzerland). Pyrene-PC was incorporated into the outer monolayer of preformed PLS3-containing proteoliposomes so that it was 10% of the lipids in the outer monolayer or 5% of the total lipids. Proteoliposomes with PLS3 were prepared by detergent dialysis, as previously described (manuscript in review). The rate of transbilayer diffusion of pyrene-10-PC was measured as previously described (Epand et al. Biochemistry 42:14576-14582 (2003), Muller et al. Chem Phys Lipids 106:89-99 (2000)). Excitation was 344 nm. When flip-flop occurs there is a reduction in the excimer emission because of dilution of the probe from one monolayer to two. The ratio of the excimer (Iₑ, emission at 476 nm) to the monomer emission (Iₘ, emission at 397 nm) was measured as a function of time before and after the addition of ionomycin, 2 mM CaCl₂ and/or apoptotic proteins. The initial ratio at time 0 was set to 1. The fluorescence experiments were carried out in 2 ml silanized glass cuvettes at 37° C. with PLS3 proteoliposomes at different lipid-to-protein ratios, and a lipid concentration of 25 μM. After addition of a freshly prepared solution of probe to the liposomes, the excimer/monomer emission ratio was monitored prior to addition of proteins or calcium until a plateau was reached. Measurements were done with two independent preparations. Values of the ratio Iₑ/Iₘ were normalized to that of the proteoliposomes alone and then averaged.

ii. Results

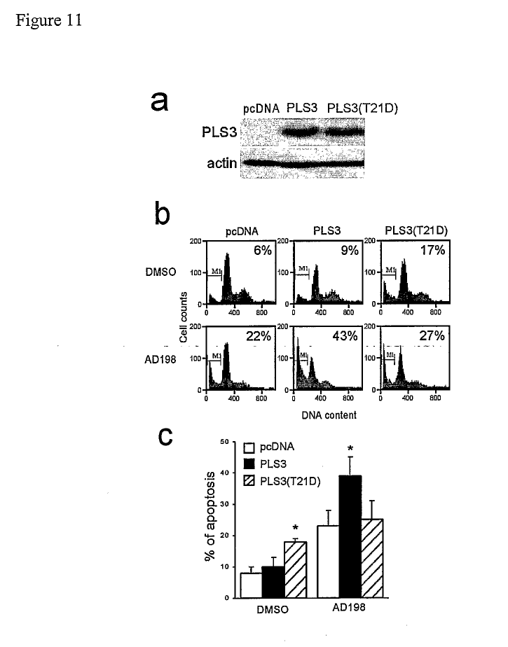

Phosphomimetic mutant of PLS3 induces apoptosis—It has been shown that overexpression of PLS3 enhanced AD198-induced apoptosis and mapped Thr21 as phosphorylation site of PLS3 by PKC-δ in AD198-induced apoptosis. In order to investigate the effect of PLS3 phosphorylation, Thr21 was mutated to the phosphomimetic residue Asp to examine whether phosphorylation of PLS3 could induce apoptosis in HeLa cells without AD198 treatment. HeLa cells were transfected with wild-type PLS3 or the phosphomimetic PLS3(T21D) mutant. Western blotting of the whole cell lysates revealed that the levels of PLS3 and PLS3(T21D) were equal (FIG. 11a). Cells were then treated with 5 μM AD198 or DMSO for 16 h. Apoptosis was analyzed with PI staining and flow cytometry. Without AD198 treatment, PLS3(T21D) induced apoptosis in 18% of HeLa cells, which is significantly higher than cells transfected with pcDNA control or wild-type PLS3 (p<0.05) (FIGS. 11b, 11c). When cells were treated with AD198 , cells expressing the wild-type PLS3 had a dramatically enhanced apoptosis (9% to 43%) compared with the HeLa-control cells (6% to 22%). In contrast, cells transfected with PLS3(T21D) had only a modest increase of apoptosis after AD198 treatment (17% to 27%) (FIGS. 11b, 11c), which is likely due to the limited transfection efficiency. This finding suggests that phosphomimetic mutant PLS3(T21D) by itself can induce apoptosis, and that phosphorylation of PLS3 at Thr21 is critical to AD198-induced apoptosis.

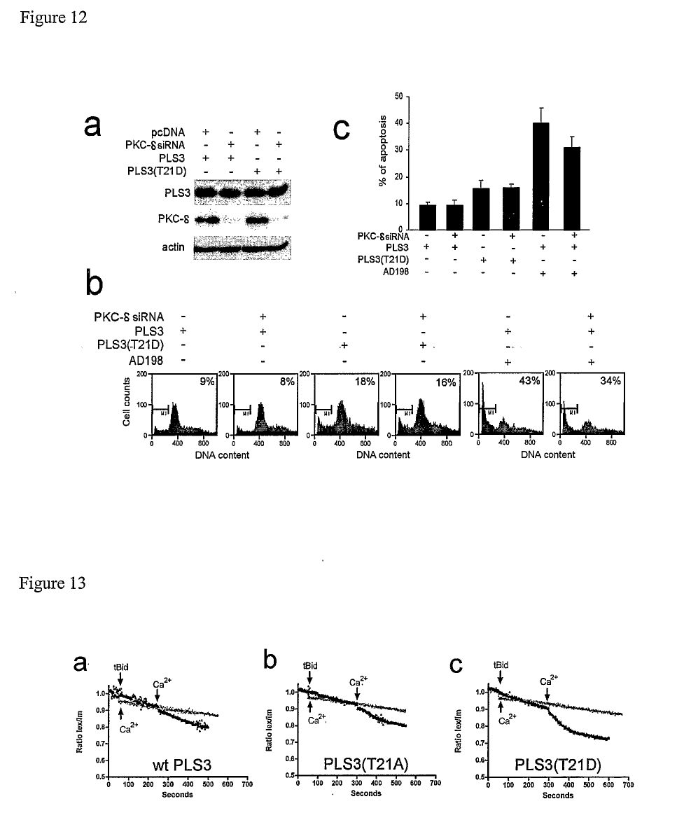

Down regulation of PKC-δ cannot suppress apoptosis induced by phosphomimetic PLS3(T21D)—Since PLS3 is a physiologic target of PKC-δ and phosphorylated at Thr21 by PKC-δ, it was examined whether down-regulation of PKC-δ can inhibit apoptosis induced by the phosphomimetic PLS3(T21D) mutant. HeLa cells were cotransfected with vectors to express PKC-δ siRNA and wild-type PLS3 or PLS3(T21D) individually or simultaneously. After 48 h, Western blot analysis of whole cell lysates from transfected cells was performed. The expression of wild-type PLS3 and PLS3(T21D) was similar and the expression of PKC-δ was decreased to less than 10% of normal by PKC-δ siRNA (FIG. 12a). Cells were then treated with 5 μM AD198 or DMSO for 16 h and harvested for apoptosis analysis. Similar to the result of FIG. 11, PLS3(T21D) induced 18% apoptosis without AD198 treatment. Down-regulation of PKC-δ by siRNA did not affect PLS3(T21D)-induced apoptosis, whereas down-regulation of PKC-δ with siRNA inhibited the enhanced apoptosis induced by wild-type PLS3 (FIGS. 12b,c). These results further support that PLS3 is a critical downstream effector of PKC-δ.

PLS3 activity is enhanced by PKC-δ phosphorylation—Next it was investigated whether phosphorylation of PLS3 by PKC-δ leads to PLS3 activation. Using proteoliposomes with a similar composition to the mitochondrial outer membrane, a phospholipid flip-flop assay was developed to measure the activity of PLS3. Similar to other members of the scramblase family, PLS3 has the capability of promoting transbilayer lipid diffusion that is calcium-dependent. When the highly conserved calcium-binding motif is mutated, the PLS3-induced phospholipid flip-flop activity is abolished. Using this assay to study the required cofactors of PLS3, it was demonstrated that tBid is another essential cofactor for PLS3 activity. The same system was used to investigate whether PLS3 activity is modulated by phosphorylation at Thr21. First proteoliposomes containing the same amounts of recombinant PLS3 or PLS3(T21D) were made. Pyrene-labeled phosphatidylcholine (pyrene-PC) was added asymmetrically to the outer leaflet of the proteoliposomes as a probe. The rate of lipid flip-flop, determined by the decrease in the ratio of excimer to monomer fluorescence emission (Ie/Im), was monitored continuously before and after sequential addition of calcium and tBid. PLS3 was first compared with PLS3 after it was phosphorylated by recombinant PKC-δ in vitro using this assay. However, a high background was detected due to the buffer used in the in vitro phosphorylation reaction. A switch was then made to recombinant PLS3(T21D) to avoid the in vitro phosphorylation reaction. As shown in FIG. 13, the decrease in the Ie/Im ratio was much more rapid and significant in PLS3(T21D) proteoliposomes than that in wild-type PLS3 proteoliposomes, confirming that the phosphomimetic mutant of PLS3 has a stronger activity than unphosphorylated PLS3. Testing the proteoliposomes reconstituted with PLS3(T21A) revealed a similar activity to that of wild-type PLS3 (FIG. 13), indicating that the phosphoinhibitory mutation did not block the activity of PLS3. It is therefore concluded that phosphorylation at Thr21 is a mechanism of PLS3 activation, in which PKC-δ induces PLS3 activation by phosphorylation at Thr21.

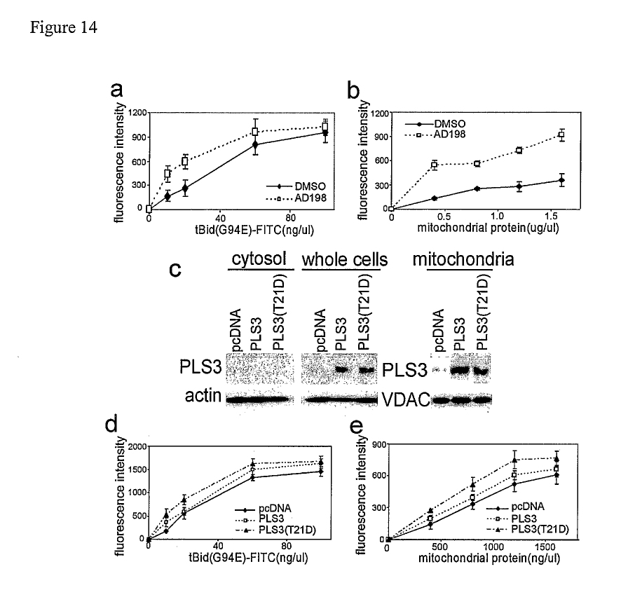

Activation of PLS3 increases CL or tBid-binding capacity on the mitochondrial surface—The downstream event in mitochondria after PLS3 is activated by PKC-δ was then examined. Quantification of CL in the mitochondrial inner and outer membranes showed that cells with overexpression of PLS3 had an increase of CL, whereas cells expressing a dominant negative mutant of PLS3 had a decrease of CL in the mitochondrial outer membrane. During apoptosis, tBid translocates to mitochondria by targeting CL (Lutter et al. (2000)). If the amount of CL on the mitochondrial surface increases, tBid targeting would be enhanced since tBid does not insert into the outer membrane devoid of surface CL. This explains why mitochondria with overexpression of PLS3 are more susceptible to tBid-induced cytochrome c release. Based on this rationale, it was investigated whether cells treated with AD198 , which activates PKC-δ to phosphorylate PLS3, had the same response as PLS3 overexpression, i.e. increased amount of CL or tBid-binding capacity on the mitochondrial surface. A fluorescent probe, FITC-tBid(G94E), was made for this study. HeLa cells were treated with 5 μM AD198 or DMSO as control for 4 h. Mitochondria were isolated from cells, and incubated with FITC-tBid(G94E). Mitochondria from AD198-treated cells had a significantly higher tBid-binding capacity than those from DMSO-treated control cells (FIGS. 14a, b). Since AD198-induced PLS3 phosphorylation at the same time point, phosphorylation of PLS3 by PKC-δ can be a contributing factor for the increase of the tBid-binding capacity. However, a concern of this interpretation is that mitochondria from AD198-treated cells may lose their mitochondrial membrane integrity and allow the FITC-tBid(G94E) probe to get access to the inner membrane CL. To test this possibility, HeLa cells were selected that were transfected with the control vector, PLS3 or PLS3(T21D) expressing vector with G418 and successfully obtained stable cell lines. Subcellular fractionation and Western bloffing showed that PLS3 and PLS3(T21D) were overexpressed in the selected clones and correctly localized in mitochondria (FIG. 14c). There was also minimal cytochrome c leaking out for mitochondria expressing PLS3(T21D), showing the maintenance of the integrity of the mitochondrial membrane. Mitochondria from the established stable cells were then used to test the tBid-binding capacity. As shown in FIGS. 14d and 14e, the mitochondria from HeLa-PLS3 cells bound more FITC-tBid(G94E) than those from HeLa-control cells, and mitochondria from HeLa-PLS3(T21D) had the highest tBid-binding capacity on the mitochondrial surface. This finding provides the in vivo evidence that PLS3(T21D) is more active than unphosphorylated PLS3.

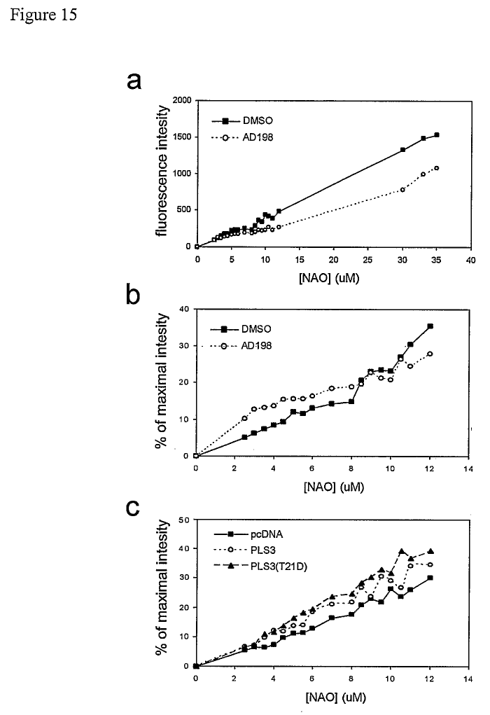

Increased CL in the mitochondrial outer membrane by phosphorylated PLS3—Because tBid was proposed to target to mitochondrial CL, the amount of CL in the mitochondrial outer membrane in cells expressing wild-type PLS3 or the PLS3(T21D) mutant was investigated. This study utilized the CL-specific dye, NAO, by titration of NAO to reach saturation with CL. CL in the outer membrane will be saturated first when the NAO concentration is gradually increased. A plateau appears when the outer membrane is saturated followed by saturation of the CL in the inner membrane (Liu et al. (2003), Garcia Fernandez, et al. (2004)). This method used cells fixed with paraformaldehyde and thus AD198-induced loss of mitochondrial integrity would not affect the result. The curves between HeLa cells incubated with or without AD198 were compared. Cells treated with AD198 always exhibited a decrease in absolute NAO fluorescence (FIG. 15a), suggesting that the amount of CL decreased after AD198 treatment. The maximal fluorescence intensity that derived from the same HeLa cells incubated with 35 μM NAO as 100% to calculate the percentage of exposed CL was then used (FIG. 15b). The curves from untreated and AD198-treated cells crossed each other at about 9 μM, which is the concentration that CL in the outer leaflet of the inner membrane began to be saturated (Liu et al. (2003), Garcia Fernandez, et al. (2004)). At concentrations lower than 9 μM, which represent CL in the outer membrane, AD198 treated cells had a higher percentage of CL than control cells. In contrast, at concentrations higher than 9 μM, which represents CL in the inner membrane, the curve of AD198-treated cells was lower (FIG. 15b). This finding suggests that CL could be translocated from the inner membrane to the outer membrane after AD198 treatment. Similar studies were also performed in mitochondria isolated from HeLa-control, HeLa-PLS3 and HeLa-PLS3(T21D) cells. The percentages of CL are always higher in HeLa-PLS3 cells than HeLa-control cells, and the curve of HeLa-PLS3(T21D) cells was even higher than in HeLa-PLS3 cells (FIG. 15c), supporting our theory that active PLS3(T21D) increases the amount of CL on the mitochondrial outer membrane.

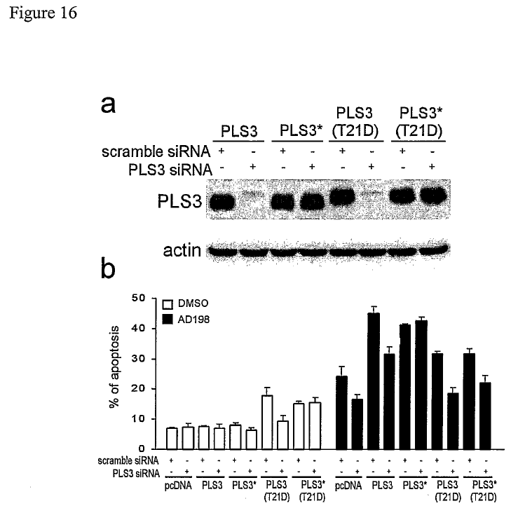

Rescuing AD198-induced apoptosis by expression of PLS3 transgenes—siRNA was used to down-regulate endogenous PLS3 and demonstrated that PLS3 was required for AD198-induced apoptosis. With the establishment of PLS3(T21D) as a more active form of PLS3, it was examined whether expression of PLS3 or PLS3(T21D) transgenes could rescue AD198-induced apoptosis when endogenous PLS3 is suppressed by siRNA. In order to avoid suppression of the transgenes by PLS3 siRNA, Gly99 (encoded by GGC) was mutated to Gly99 (GGA), which is localized at the center of PLS3-siRNA sequence. HeLa cells were transfected with scrambled control siRNA or PLS3 siRNA or PLS3 expressing vectors. Western blotting of the whole cell lysates with PLS3 antibody was used to test this strategy. PLS3 siRNA suppressed the expression of wild-type PLS3 and PLS3(T21D) transgenes if their sequences fully correspond to PLS3 siRNA. In contrast, the Gly99 mutants, designated by PLS3* and PLS3(T21D)*, which contains the C→A mutation, cannot be suppressed by PLS3 siRNA (FIG. 16a). Their sensitivity to AD198-induced apoptosis was then examined. Cells were treated with 5 μM AD198 or DMSO for 16 h. Apoptosis was analyzed by PI staining. Consistent with the result in FIG. 11, PLS3(T21D) by itself could induce small degree of apoptosis without AD198 treatment, and this effect is suppressed by PLS3 siRNA as the expression of PLS3(T21D) was down-regulated. If cells expressed PLS3(T21D)*, same degree of apoptosis was detected, and PLS3 siRNA could not decrease the level of PLS3(T21D)* nor inhibit apoptosis. With AD198 treatment, overexpression of PLS3 enhanced apoptosis compared with cells transfected with the control pcDNA vector. This enhancement could be suppressed by PLS3 siRNA. However, the enhancing effect from overexpression of PLS3* was not inhibited by PLS3 siRNA. Finally, AD198 treatment increased apoptosis in cells expressing PLS3(T21D) or PLS3(T21D)*, which is likely due to the limited efficiency in transfection. This is supported by the observation that co-transfection with PLS3 siRNA decreased the level of apoptosis to the same as cells transfected with PLS3(T21D), indicating that the additional apoptosis after AD198 treatment is due to apoptosis from cells that did not incorporate the PLS3(T21D) expressing vector.

iii. Discussion

PLS3 is phosphorylated by PKC-δ at Thr21, and PLS3 is the mitochondrial target of PKC-δ-induced apoptosis (Liu et al. (2003)). It was investigated whether phosphorylation of PLS3 at Thr21 leads to PLS3 activation and what are the downstream events of PLS3 activation. To study the effect of PLS3 phosphorylation, a phosphomimetic mutant PLS3(T21D) was constructed. Even though PLS3 is likely a pro-apoptotic factor, cells expressing wild-type PLS3 remain viable, which is most likely due to a low baseline PLS3 activity. However, overexpression of PLS3 significantly enhanced apoptosis induced by AD198, a PKC-δ activator (Roaten et al. Mol Cancer Ther, 1:483-492 (2002)) that induces PLS3 phosphorylation by PKC-δ at Thr21. This is in contrast to cells expressing PLS3(T21D), which have a higher degree of apoptosis without AD198 treatment, but no enhanced apoptosis after AD198 treatment. Down-regulation of PKC-δ with siRNA suppressed AD198-induced apoptosis in both control cells and cells expressing wild-type PLS3, but not in cells expressing PLS3(T21D). These findings are consistent with the notion that PLS3 is a downstream effector of PKC-δ-induced apoptosis.

The next question is how phosphorylation of PLS3 by PKC-δ modulates PLS3 activity. In order to test whether phosphorylation of PLS3 induces its activation, an enzymatic assay (Epand et al. Biochemistry 42:14576-14582 (2003)) was used, based on the phospholipid flip-flop activity of PLS3. Recombinant protein of the phosphomimetic mutant PLS3(T21D) was generated to avoid the step of in vitro PKC-δ phosphorylation. This strategy worked and recombinant PLS3(T21D) was shown to be more active than the unphosphorylated wild-type PLS3. The activity of PLS3(T21A), the phosphorylation-inhibited mutant, was also examined, and found to be no different from that of wild-type PLS3, indicating that inhibition of phosphorylation does not abolish the baseline activity of PLS3. HeLa cells expressing the PLS3(T21A) could not enhance AD198-induced apoptosis like wild-type PLS3. These findings prove that PLS3 activity is regulated by PKC-δ-induced phosphorylation.

After we establish that PLS3 is phosphorylated and activated by PKC-δ, the consequence of PLS3 activation and how that event induces apoptosis was examined. Based on findings in CL distribution in cells expressing wild-type PLS3 or the inactive PLS3(F258V) mutant that abolishes the calcium-binding motif of PLS3, one possibility is that overexpression of PLS3 or activation of PLS3 increases the amount of CL on the mitochondrial surface to facilitate tBid targeting. Thus, FITC-tBid(G94E) were generated to quantify the tBid-binding capacity on the mitochondrial surface. This probe was developed based on an important assumption that tBid does not insert into the membrane but has a parallel orientation to the membrane surface as described by two independent NMR studies (Gong et al. J Biol Chem, 279:28954-28960 (2004), Oh et al. J Biol Chem, 280:753-767 (2005)). Based on these two independent observations that tBid does not insert into the membrane, the target of tBid, presumably CL, must be present on the surface of mitochondria to be accessible by tBid. The FITC-tBid(G94E) probe will not penetrate the outer membrane to interact with CL in the mitochondrial inner membrane and therefore could be used to quantify CL on the mitochondrial surface. The reason to use tBid(G94E) rather than tBid is its inability to induce mitochondrial damage due to the defective BH3 domain (Luo et al. Cell 94:481-490 (1998)). Another advantage is that the probe mimics tBid targeting and is thus better than using the antibody against CL as a probe. Other potential related tBid targets, such as monolysocardiolipin (MLCL) (Esposti et al. Cell Death Differ 10:1300-1309 (2003), Degli Esposti et al. Ital J Biochem 52:43-50 (2003)), could also bind the FITC-tBid(G94E) probe, and were included in the derived tBid-binding capacity.

The hypothesis that PLS3 is responsible for translocating CL to the mitochondrial surface was tested by quantification of tBid-binding capacity on the mitochondrial surface. Mitochondria from cells treated with AD198 had a higher tBid-binding capacity than untreated cells. Since this increase could be due to damage of the mitochondrial outer membrane in AD198-induced apoptotic cells, mitochondria isolated from cells expressing PLS3(T21D) or wild-type PLS3 was examined. Mitochondria with expression of PLS3(T21D) had the highest tBid-binding capacity, and those expressing PLS3 exhibited a tBid-binding capacity higher than control mitochondria. These results are consistent with PLS3 translocating CL to the mitochondrial surface. 287. As an alternative approach, CL-specific fluorescent dye NAO was used to quantify the total amount and the distribution of CL in the outer and inner membranes. This method was used by Garcia-Fernadez et al. (Garcia Fernandez et al. (2002), Garcia Fernandez et al. FEBS Lett, 478:290-294 (2000)) to study changes of CL in the early phase of apoptosis. The methodology is based on the unique characteristic of NAO in binding CL at two types of stoichiometry. At 1:1 binding, NAO has fluorescence emission at 530 nm; whereas at 2:1 binding, NAO has emission at 590 nm. Accurate quantification of CL by NAO requires saturation of CL with NAO. By plotting the fluorescence intensity at 590 nm versus the NAO concentrations, Garcia-Fernandez et al. showed that there was a plateau when cells were incubated with 10 μM NAO (Garcia Fernandez et al. (2002), Garcia Fernandez et al. (2000)), which represented the concentration at which CL in the outer leaflet of mitochondrial inner membrane became saturated. The methodology was refined, and the same principle used to determine the relative percentage of CL in the outer membrane by incubating cells with NAO at concentrations lower than 10 μM. At this range of NAO concentrations, the curve of fluorescence intensity relative to the maximal intensity for cells expressing PLS3(T21D) is always higher than those expressing PLS3, which is also higher than control cells. Cells treated with AD198 also exhibited higher percentages relative to the maximal intensity compared with cells treated with DMSO control. The supports the notion that PLS3 is responsible for increasing CL in the outer membrane of mitochondria, and phosphorylation of PLS3 at Thr21 will further induce PLS3 activation. Because CL is synthesized in the inner membrane of mitochondria (Schlame et al. Prog Lipid Res 39:257-288 (2000)), PLS3 is thus likely the trafficking machinery for moving CL to the outer membrane of mitochondria. CL in the outer membrane is mainly in the contact zone, which is the location where the outer and inner membranes have the closest contact, and would be the natural location when CL is translocated to the outer membrane. The electron microscopic study by Lutter et al. also reported that tBid(G94E) is localized in the contact zone (Lutter et al. BMC Cell Biol 2: 22 (2001)).

In conclusion, it is shown that phosphorylation of PLS3 by PKC-δ leads to activation of PLS3 by an in vitro PLS3 enzymatic assay and by the in vivo analysis of PLS3-induced CL changes. These findings support the importance of PLS3 as the downstream effector in PKC-δ-induced apoptosis.

Throughout this application, various publications are referenced. The disclosures of these publications in their entireties are hereby incorporated by reference into this application in order to more fully describe the state of the art to which this invention pertains.

### J. REFERENCES

Adams, J. M. and S. Cory Life-or-death decisions by the Bcl-2 protein family. Trends Biochem Sci 26:61-6 (2001).

al-Khodairy, F., E. Fotou, K. S. Sheldrick, D. J. Griffiths, A. R. Lehmann, and A. M. Carr. Identification and characterization of new elements involved in checkpoint and feedback controls in fission yeast. Mol Biol Cell 5:147-60 (1994).

An W G, Hwang S G, Trepel J B, Blagosklonny M V. Protease inhibitor-induced apoptosis: accumulation of wt p 53, p 21 WAF1/CIP1, and induction of apoptosis are independent markers of proteasome inhibition. Leukemia 14:1276-1283 (2000).

Barrett, C. M., F. L. Lewis, et al. Novel extranuclear-targeted anthracyclines override the antiapoptotic functions of Bcl-2 and target protein kinase C pathways to induce apoptosis. Mol Cancer Ther 1: 469-81 (2002).

Baud, V. and Karin, M. Signal transduction by tumor necrosis factor and its relatives. Trends Cell Biol 11:372-377 (2001).

Ben-Efraim, I., Zhou, Q., Wiedmer, T., Gerace, L., and Sims, P. J. Phospholipid scramblase 1 is imported into the nucleus by a receptor-mediated pathway and interacts with DNA. Biochemistry 43:3518-3526 (2004).

Bevers, E. M., Comfurius, P., Dekkers, D. W., and Zwaal, R. F. Lipid translocation across the plasma membrane of mammalian cells. Biochim Biophys Acta, 1439:317-330 (1999).

Bevers, E. M., P. Comfurius, D. W. Dekkers, M. Harmsma, and R. F. Zwaal Transmembrane phospholipid distribution in blood cells: control mechanisms and pathophysiological significance. Biol Chemn 379:973-86 (1998).

Bilyeu, J. D., Panta, G. R., Cavin, L. G., Barrett, C. M., Turner, E. J., Sweatman, T. W., Israel, M., Lothstein, L., and Arsura, M. Circumvention of nuclear factor kappaB-induced chemoresistance by cytoplasmic-targeted anthracyclines. Mol Pharmacol 65:1038-1047 (2004).

Blass, M., Kronfeld, I., Kazimirsky, G., Blumberg, P. M., and Brodie, C. Tyrosine phosphorylation of protein kinase Cdelta is essential for its apoptotic effect in response to etoposide. Mol Cell Biol 22:182-195 (2002).

Brodie, C. and Blumberg, P. M. Regulation of cell apoptosis by protein kinase c delta. Apoptosis 8:19-27 (2003).

Chen J, Freeman A, Liu J, Dai Q, Lee R M. The apoptotic effect of HA14-1, a Bcl-2-interacting small molecular compound, requires Bax translocation and is enhanced by PK11195. Mol Cancer Ther. 1:961-967 (2002).

Chen, M. J., Y. T. Lin, H. B. Lieberman, G. Chen, and E. Y. Lee. ATM-dependent phosphorylation of human Rad9 is required for ionizing radiation-induced checkpoint activation. J Biol Chem 276:16580-6 (2001).

Clark, A. S., K. A. West, P. M. Blumberg, and P. A. Dennis. Altered protein kinase C (PKC) isoforms in non-small cell lung cancer cells: PKCdelta promotes cellular survival and chemotherapeutic resistance. Cancer Res 63:780-6 (2003).

Cory, S. and J. M. Adams. Matters of life and death: programmed cell death at Cold Spring Harbor. Biochim Biophys Acta 1377:R25-44 (1998).

Cristea, I. M. and Degli Esposti, M. Membrane lipids and cell death: an overview. Chem Phys Lipids 129: 133-160 (2004).

Crosby, D. and A. W. Poole. Physical and functional interaction between protein kinase C delta and Fyn tyrosine kinase in human platelets. J Biol Chem 278:24533-41 (2003).

Degli Esposti, M. The mitochondrial battlefield and membrane lipids during cell death signalling. Ital J Biochem, 52:43-50 (2003).

Denning, M. F., Wang, Y., Tibudan, S., Alkan, S., Nickoloff, B. J., and Qin, J. Z. Caspase activation and disruption of mitochondrial membrane potential during UV radiation-induced apoptosis of human keratinocytes requires activation of protein kinase C. Cell Death Differ 9:40-52 (2002).

Denning, M. F., Y. Wang, B. J. Nickoloff, and T. Wrone-Smith. Protein kinase Cdelta is activated by caspase-dependent proteolysis during ultraviolet radiation-induced apoptosis of human keratinocytes. J Biol Chem 273:29995-30002 (1998).

Desagher, S., Osen-Sand, A., Nichols, A., Eskes, R., Montessuit, S., Lauper, S., Maundrell, K., Antonsson, B., and Martinou, J. C. Bid-induced conformational change of Bax is responsible for mitochondrial cytochrome c release during apoptosis. J Cell Biol, 144: 891-901 (1999).

DeVries, T. A., Neville, M. C., and Reyland, M. E. Nuclear import of PKCdelta is required for apoptosis: identification of a novel nuclear import sequence. Embo J, 21:6050-6060 (2002).

Druker, B. J., M. Talpaz, D. J. Resta, B. Peng, E. Buchdunger, J. M. Ford, N. B. Lydon, H. Kantarjian, R. Capdeville, S. Ohno-Jones, and C. L. Sawyers. Efficacy and safety of a specific inhibitor of the BCR-ABL tyrosine kinase in chronic myeloid leukemia. N Engl J Med 344:1031-7 (2001).

Du, C., Fang, M., Li, Y., Li, L., and Wang, X. Smac, a mitochondrial protein that promotes cytochrome c-dependent caspase activation by eliminating IAP inhibition [In Process Citation]. Cell 102: 33-42 (2000).

Emoto, Y., Manome, Y., Meinhardt, G., Kisaki, H., Kharbanda, S., Robertson, M., Ghayur, T., Wong, W. W., Kamen, R., Weichselbaum, R., and et al. Proteolytic activation of protein kinase C delta by an ICE-like protease in apoptotic cells. Embo J 14:6148-6156 (1995).

Epand, R. F., Martinou, J. C., Montessuit, S., and Epand, R. M. Transbilayer lipid diffusion promoted by bax: implications for apoptosis. Biochemistry, 42: 14576-14582, (2003).

Esposti, M. D., Cristea, I. M., Gaskell, S. J., Nakao, Y., and Dive, C. Proapoptotic Bid binds to monolysocardiolipin, a new molecular connection between mitochondrial membranes and cell death. Cell Death Differ, 10:1300-1309 (2003).

Eskes, R., Desagher, S., Antonsson, B., and Martinou, J. C. Bid induces the oligomerization and insertion of Bax into the outer mitochondrial membrane. Mol Cell Biol, 20:929-935 (2000).

Evan, G. and Littlewood, T. A matter of life and cell death. Science 281:1317-1322, (1998).

Fadok, V. A., Voelker, D. R., Campbell, P. A., Cohen, J. J., Bratton, D. L., and Henson, P. M. Exposure of phosphatidylserine on the surface of apoptotic lymphocytes triggers specific recognition and removal by macrophages. J Immunol 148:2207-2216 (1992).

Frasch, S. C., Henson, P. M., Kailey, J. M., Richter, D. A., Janes, M. S., Fadok, V. A., and Bratton, D. L. Regulation of phospholipid scramblase activity during apoptosis and cell activation by protein kinase Cdelta. J Biol Chem 275:23065-23073 (2000).

Garcia Fernandez, M. I., Ceccarelli, D., and Muscatello, U. Use of the fluorescent dye 10-N-nonyl acridine orange in quantitative and location assays of cardiolipin: a study on different experimental models. Anal Biochem, 328:174-180 (2004).

Garcia Fernandez, M., Troiano, L., Moretti, L., Nasi, M., Pinti, M., Salvioli, S., Dobrucki, J., and Cossarizza, A. Early changes in intramitochondrial cardiolipin distribution during apoptosis. Cell Growth Differ, 13: 449-455 (2002).

Garcia Fernandez, M., Troiano, L., Moretti, L., Pedrazzi, J., Salvioli, S., Castilla-Cortazar, I., and Cossarizza, A. Changes in intramitochondrial cardiolipin distribution in apoptosis-resistant HCW-2 cells, derived from the human promyelocytic leukemia HL-60. FEBS Lett, 478:290-294 (2000).

Ghayur, T., M. Hugunin, R. V. Talanian, S. Ratnofsky, C. Quinlan, Y. Emoto, P. Pandey, R. Datta, Y. Huang, S. Kharbanda, H. Allen, R. Kamen, W. Wong, and D.

Green, D. R. and Evan, G. I. A matter of life and death. Cancer Cell 1:19-30 (2002).

Gong, X. M., Choi, J., Franzin, C. M., Zhai, D., Reed, J. C., and Marassi, F. M. Conformation of membrane-associated proapoptotic tBid. J Biol Chem. 279:28954-28960 (2004).

Green, D. R. and G. I. Evan. A matter of life and death. Cancer Cell 1:19-30 (2002).

Harstrick, A., Vanhoefer, U., Schleucher, N., Schroeder, J., Baumgart, J., Scheulen, M. E., Wilke, H., and Seeber, S. Activity of N-benzyl-adriamycin-14-valerate (AD198 ), a new anthracycline derivate, in multidrug resistant human ovarian and breast carcinoma cell lines. Anticancer Drugs, 6:681-685 (1995).

Hofmann, J. Modulation of protein kinase C in antitumor treatment. Rev Physiol Biochem Pharmacol 142:1-96 (2001).

Ito, Y., N. C. Mishra, K. Yoshida, S. Kharbanda, S. Saxena, and D. Kufe Mitochondrial targeting of JNK/SAPK in the phorbol ester response of myeloid leukemia cells. Cell Death Differ 8:794-800 (2001).

Ito, Y., P. Pandey, N. Mishra, S. Kumar, N. Narula, S. Kharbanda, S. Saxena, and D. Kufe (2001) Targeting of the c-Ab1 Tyrosine Kinase to Mitochondria in Endoplasmic Reticulum Stress-Induced Apoptosis. Mol Cell Biol 21:6233-42.

Joseloff, E., C. Cataisson, H. Aamodt, H. Ocheni, P. Blumberg, A. J. Kraker, S. H. Yuspa Src family kinases phosphorylate protein kinase C delta on tyrosine residues and modify the neoplastic phenotype of skin keratinocytes. J Biol Chem 277:12318-23 (2002).

Kerr, J. F., Wyllie, A. H., and Currie, A. R. Apoptosis: a basic biological phenomenon with wide-ranging implications in tissue kinetics. Br J Cancer 26:239-257 (1972).

Komatsu, K., T. Miyashita, H. Hang, K. M. Hopkins, W. Zheng, S. Cuddeback, M. Yamada, H. B. Lieberman, and H. G. Wang. Human homologue of S. pombe Rad9 interacts with BCL-2/BCL-xL and promotes apoptosis. Nat Cell Biol 2:1-6 (2000).

Konishi, H., E. Yamauchi, H. Taniguchi, T. Yamamoto, H. Matsuzaki, Y. Takemura, K. Ohmae, U. Kikkawa, and Y. Nishizuka. Phosphorylation sites of protein kinase C delta in H2O2-treated cells and its activation by tyrosine kinase in vitro. Proc Natl Acad Sci USA 98: 6587-92 (2001).

Korsmeyer, S. J., Wei, M. C., Saito, M., Weiler, S., Oh, K. J., and Schlesinger, P. H. Pro-apoptotic cascade activates BID, which oligomerizes BAK or BAX into pores that result in the release of cytochrome c. Cell Death Differ, 7:1166-1173 (2000).

Krammer, P. H. CD95's deadly mission in the immune system. Nature 407:789-795, (2000).

Kufe. Proteolytic activation of protein kinase C delta by an ICE/CED 3-like protease induces characteristics of apoptosis. J Exp Med 184:2399-404 (1996).

Kumar, S., N. Mishra, D. Raina, S. Saxena, and D. Kufe. Abrogation of the cell death response to oxidative stress by the c-Ab1 tyrosine kinase inhibitor STI571. Mol Pharmacol 63:276-82 (2003).

Kuwana, T., Mackey, M. R., Perkins, G., Ellisman, M. H., Latterich, M., Schneiter, R., Green, D. R., and Newmeyer, D. D. Bid, bax, and lipids cooperate to form supramolecular openings in the outer mitochondrial membrane. Cell 111: 331-342 (2002).

LeBlanc R, Catley L P, Hideshima T, Lentzsch S, Mitsiades C S, Mitsiades N, Neuberg D, Goloubeva O, Pien C S, Adams J, Gupta D, Richardson P G, Munshi N C, Anderson K C. Proteasome inhibitor PS-341 inhibits human myeloma cell growth in vivo and prolongs survival in a murine model. Cancer Res. 62:4996-5000 (2002).

Li, L. Y., Luo, X., and Wang, X. Endonuclease G is an apoptotic DNase when released from mitochondria. Nature 412: 95-99 (2001).

Li, L., P. S. Lorenzo, K. Bogi, P. M. Blumberg, and S. H. Yuspa. Protein kinase Cdelta targets mitochondria, alters mitochondrial membrane potential, and induces apoptosis in normal and neoplastic keratinocytes when overexpressed by an adenoviral vector. Mol Cell Biol 19:8547-58 (1999).

Li, P., Nijhawan, D., Budihardjo, I., Srinivasula, S. M., Ahmad, M., Alnemri, E. S., and Wang, X. Cytochrome c and dATP-dependent formation of Apaf-1/caspase-9 complex initiates an apoptotic protease cascade. Cell 91:479-489 (1997).

Ling Y H, Liebes L, Zou Y, Perez-Soler R. Reactive oxygen species generation and mitochondrial dysfunction in the apoptotic response to bortezomib, a novel proteasome inhibitor, in human H460 non-small cell lung cancer cells. J Biol Chem. 278:33714-33723 (2003)

Liu, J., Weiss, A., Durrant, D., Chi, N. W., and Lee, R. M. The cardiolipin-binding domain of Bid affects mitochondrial respiration and enhances cytochrome c release. Apoptosis, 9:533-541 (2004).

Liu, J., Dai, Q., Chen, J., Durrant, D., Freeman, A., Liu, T., Grossman, D., and Lee, R. M. Phospholipid scramblase 3 controls mitochondrial structure, function, and apoptotic response. Mol Cancer Res 1:892-902 (2003).

Liu, J., Chen, J., Dai, Q., and Lee, R. M. Phospholipid Scramblase 3 Is the Mitochondrial Target of Protein Kinase C delta-induced Apoptosis. Cancer Res. 63:1153-1156 (2003).

Liu, J., Durrant, D., and Lee, R. M. Protein kinase C-delta and its downstream effectors as potential targets for cancer therapy. Cancer Therapy 1:289-295 (2003).

Lothstein, L., Rodrigues, P. J., Sweatman, T. W., and Israel, M. Cytotoxicity and intracellular biotransformation of N-benzyladriamycin-14-valerate (AD 198) are modulated by changes in 14-O-acyl chain length. Anticancer Drugs 9:58-66 (1998).

Lothstein, L., Koseki, Y., and Sweatman, T. W. P-glycoprotein overexpression in mouse cells does not correlate with resistance to N-benzyladriamycin-14-valerate (AD 198). Anticancer Drugs 5:623-633 (1994).

Lothstein, L., Sweatman, T. W., Dockter, M. E., and Israel, M. Resistance to N-benzyladriamycin-14-valerate in mouse J774.2 cells: P-glycoprotein expression without reduced N-benzyladriamycin-14-valerate accumulation. Cancer Res. 52:3409-3417 (1992).

Lothstein, L., Wright, H. M., Sweatman, T. W., and Israel, M. N-benzyladriamycin-14-valerate and drug resistance: correlation of anthracycline structural modification with intracellular accumulation and distribution in multidrug resistant cells. Oncol Res 4:341-347 (1992).

Lu, Z., D. Liu, A. Hornia, W. Devonish, M. Pagano, and D. A. Foster. Activation of protein kinase C triggers its ubiquitination and degradation. Mol Cell Biol 18:839-45 (1998).

Luo, X., Budihardjo, I., Zou, H., Slaughter, C., and Wang, X. Bid, a Bcl2 interacting protein, mediates cytochrome c release from mitochondria in response to activation of cell surface death receptors. Cell, 94: 481-490 (1998).

Lutter, M., M. Fang, X. Luo, M. Nishijima, X. Xie, and X. Wang. Cardiolipin provides specificity for targeting of tBid to mitochondria. Nat Cell Biol 2:754-761 (2000).

Lutter, M., Perkins, G. A., and Wang, X. The pro-apoptotic Bcl-2 family member tBid localizes to mitochondrial contact sites. BMC Cell Biol, 2:22 (2001).

McCracken, M. A., L. J. Miraglia, R. A. McKay, and J. S. Strobl Protein kinase C delta is a prosurvival factor in human breast tumor cell lines. Mol Cancer Ther 2:273-81.

McMillin, J. B. and Dowhan, W. Cardiolipin and apoptosis. Biochim Biophys Acta, 1585: 97-107 (2002).

Majumder, P. K., N. C. Mishra, X. Sun, A. Bharti, S. Kharbanda, S. Saxena, and D. Kufe. Targeting of protein kinase C delta to mitochondria in the oxidative stress response. Cell Growth Differ 12:465-70 (2001).

Majumder, P. K., P. Pandey, X. Sun, K. Cheng, R. Datta, S. Saxena, S. Kharbanda, and D. Kufe. Mitochondrial translocation of protein kinase C delta in phorbol ester-induced cytochrome c release and apoptosis. J Biol Chem 275:21793-6 (2000).

Mandil, R., E. Ashkenazi, M. Blass, I. Kronfeld, G. Kazimirsky, G. Rosenthal, F. Umansky, P. S. Lorenzo, P. M. Blumberg, and C. Brodie. Protein kinase Calpha and protein kinase Cdelta play opposite roles in the proliferation and apoptosis of glioma cells. Cancer Res 61: 4612-9 (2001).

Marshall, J. L., N. Bangalore, D. El-Ashry, Y. Fuxman, M. Johnson, B. Norris, M. Oberst, E. Ness, S. Wojtowicz-Praga, P. Bhargava, N. Rizvi, S. Baidas, and M. J. Hawkins. Phase I study of prolonged infusion Bryostatin-1 in patients with advanced malignancies. Cancer Biol Ther 1:409-16 (2002).

Mecklenbrauker, I., K. Saijo, N. Y. Zheng, M. Leitges, and A. Tarakhovsky Protein kinase Cdelta controls self-antigen-induced B-cell tolerance. Nature 416:860-5 (2002).

Minoffi, G., Menna, P., Salvatorelli, E., Cairo, G., and Gianni, L. Anthracyclines: molecular advances and pharmacologic developments in antitumor activity and cardiotoxicity. Pharmacol Rev. 56:185-229 (2004).

Mitsiades N, Mitsiades C S, Poulaki V, Chauhan D, Fanourakis G, Gu X, Bailey C, Joseph M, Libermann T A, Treon S P, Munshi N C, Richardson P G, Hideshima T, Anderson K C. Molecular sequelae of proteasome inhibition in human multiple myeloma cells. Proc Natl Acad Sci USA.; 99:14374-14379 (2002).

Miyamoto, A., K. Nakayama, H. Imaki, S. Hirose, Y. Jiang, M. Abe, T. Tsukiyama, H. Nagahama, S. Ohno, S. Hatakeyama, and K. I. Nakayama Increased proliferation of B cells and auto-immunity in mice lacking protein kinase Cdelta. Nature 416:865-9 (2002).

Muller, P., Schiller, S., Wieprecht, T., Dathe, M., and Herrmann, A. Continuous measurement of rapid transbilayer movement of a pyrene-labeled phospholipid analogue. Chem Phys Lipids, 106: 89-99 (2000).

Nishizuka, Y. Intracellular signaling by hydrolysis of phospholipids and activation of protein kinase C. Science 258:607-614 (1992).

Oh, K. J., Barbuto, S., Meyer, N., Kim, R. S., Collier, R. J., and Korsmeyer, S. J. Conformational changes in BID, a pro-apoptotic BCL-2 family member, upon membrane binding. A site-directed spin labeling study. J Biol Chem. 280:753-767 (2005).

O'Dwyer, M. E., M. J. Mauro, and B. J. Druker STI571 as a targeted therapy for CML. Cancer Invest 21: 429-38 (2003).

Ohno, S. The distinct Biological Potential of PKC Isotypes. In: L. D. Peter Parker (ed.), Protein kinase C: R.G. Landes Company, (1997).

Palombella V J, Rando O J, Goldberg A L, Maniatis T. The ubiquitin-proteasome pathway is required for processing the NF-kappa B1 precursor protein and the activation of NF-kappa B. Cell. 78:773-785 (1994).

Parker, P. J. and Dekker, L. V., Protein kinase C. R. G. Landers Company (1997).

Parrish, J., Li, L., Klotz, K., Ledwich, D., Wang, X., and Xue, D. Mitochondrial endonuclease G is important for apoptosis in C. elegans. Nature 412:90-94 (2001).

Pei X Y, Dai Y, Grant S. The proteasome inhibitor bortezomib promotes mitochondrial injury and apoptosis induced by the small molecule Bcl-2 inhibitor HA14-1 in multiple myeloma cells. Leukemia. 17:2036-2045 (2003).

Read M A, Neish A S, Luscinskas F W, Palombella V J, Maniatis T, Collins T. The proteasome pathway is required for cytokine-induced endothelial-leukocyte adhesion molecule expression. Immunity. 2:493-506 (1995).

Reed, J. C. (1999) Dysregulation of apoptosis in cancer. J Clin Oncol 17:2941-53.

Reyland, M. E., S. M. Anderson, A. A. Matassa, K. A. Barzen, and D. O. Quissell Protein kinase C delta is essential for etoposide-induced apoptosis in salivary gland acinar cells. J Biol Chem 274:19115-23 (1999).

Roaten, J. B., Kazanietz, M. G., Caloca, M. J., Bertics, P. J., Lothstein, L., Parrill, A. L., Israel, M., and Sweatman, T. W. Interaction of the novel anthracycline antitumor agent N-benzyladriamycin-14-valerate with the C1-regulatory domain of protein kinase C: structural requirements, isoform specificity, and correlation with drug cytotoxicity. Mol Cancer Ther. 1:483-492 (2002).

Roaten, J. B., Kazanietz, M. G., Sweatman, T. W., Lothstein, L., Israel, M., and Parrill, A. L. Molecular models of N-benzyladriamycin-14-valerate (AD 198) in complex with the phorbol ester-binding C1b domain of protein kinase C-delta. J Med Chem 44:1028-1034 (2001).

Roychowdhury, D. and M. Lahn. Antisense therapy directed to protein kinase C-alpha (Affinitak, LY900003/ISIS 3521): potential role in breast cancer. Semin Oncol 30:30-3 (2003).

Scheel-Toellner, D., D. Pilling, A. N. Akbar, D. Hardie, G. Lombardi, M. Salmon, and J. M. Lord Inhibition of T cell apoptosis by IFN-beta rapidly reverses nuclear translocation of protein kinase C-delta. Eur J Immunol 29:2603-12 (1999).

Schlame, M., Rua, D., and Greenberg, M. L. The biosynthesis and functional role of cardiolipin. Prog Lipid Res, 39: 257-288 (2000).

Sims, P. J. and T. Wiedmer Unraveling the mysteries of phospholipid scrambling. Thromb Haemost 86:266-75 (2001).

Sorice, M., Circella, A., Cristea, I. M., Garofalo, T., Di Renzo, L., Alessandri, C., Valesini, G., and Esposti, M. D. Cardiolipin and its metabolites move from mitochondria to other cellular membranes during death receptor-mediated apoptosis. Cell Death Differ 11:1133-1145 (2004).

Sun, J., J. Zhao, M. A. Schwartz, J. Y. Wang, T. Wiedmer, and P. J. Sims c-Ab1 tyrosine kinase binds and phosphorylates phospholipid scramblase 1. J Biol Chem 276:28984-90 (2001).

Sun, X., F. Wu, R. Datta, S. Kharbanda, and D. Kufe Interaction between protein kinase C delta and the c-Ab1 tyrosine kinase in the cellular response to oxidative stress. J Biol Chem 275:7470-3 (2000).

Sun, X., P. Majumder, H. Shioya, F. Wu, S. Kumar, R. Weichselbaum, S. Kharbanda, and D. Kufe Activation of the cytoplasmic c-Ab1 tyrosine kinase by reactive oxygen species. J Biol Chem 275:17237-40 (2000).

Susin, S. A., Lorenzo, H. K., Zamzami, N., Marzo, I., Snow, B. E., Brothers, G. M., Mangion, J., Jacotot, E., Costantini, P., Loeffler, M., Larochette, N., Goodlett, D. R., Aebersold, R., Siderovski, D. P., Penninger, J. M., and Kroemer, G. Molecular characterization of mitochondrial apoptosis-inducing factor. Nature 397:441-446 (1999).

Swannie, H. C. and S. B. Kaye Protein kinase C inhibitors. Curr Oncol Rep 4:37-46 (2002).

Verhagen, A. M., Ekert, P. G., Pakusch, M., Silke, J., Connolly, L. M., Reid, G. E., Moritz, R. L., Simpson, R. J., and Vaux, D. L. Identification of DIABLO, a mammalian protein that promotes apoptosis by binding to and antagonizing IAP proteins [In Process Citation]. Cell 102: 43-53 (2000).

Volkmer, E. and Karnitz, L. M. Human homologs of Schizosaccharomyces pombe rad1, hus1, and rad9 form a DNA damage-responsive protein complex. J Biol Chem 274:567-570 (1999).

Wang, X. The expanding role of mitochondria in apoptosis. Genes Dev 15:2922-2933 (2001).

Wang, X. Y., E. Repasky, and H. T. Liu. Antisense inhibition of protein kinase Calpha reverses the transformed phenotype in human lung carcinoma cells. Exp Cell Res 250:253-63 (1999).

Wiedmer, T., Zhao, J., Li, L., Zhou, Q., Hevener, A., Olefsky, J. M., Curtiss, L. K., and Sims, P. J. Adiposity, dyslipidemia, and insulin resistance in mice with targeted deletion of phospholipid scramblase 3 (PLSCR3). Proc Natl Acad Sci USA 101:13296-13301 (2004).

Wiedmer, T., Zhao, J., Nanjundan, M., and Sims, P. J. Palmitoylation of phospholipid scramblase 1 controls its distribution between nucleus and plasma membrane. Biochemistry 42:1227-1233 (2003).

Wiedmer, T., Zhou, Q., Kwoh, D. Y., and Sims, P. J. Identification of three new members of the phospholipid scramblase gene family. Biochim Biophys Acta 1467:244-253 (2000).

Williamson, P. and Schlegel, R. A. Transbilayer phospholipid movement and the clearance of apoptotic cells. Biochim Biophys Acta 1585:53-63 (2002).

Yoshida, K., Wang, H. G., Miki, Y., and Kufe, D. Protein kinase Cdelta is responsible for constitutive and DNA damage-induced phosphorylation of Rad9. Embo J. 22:1431-1441 (2003).

Yoshida, K., K. Komatsu, H. G. Wang, and D. Kufe c-Ab1 tyrosine kinase regulates the human Rad9 checkpoint protein in response to DNA damage. Mol Cell Biol 22:3292-300 (2002).

Yoshida, K., Y. Miki, and D. Kufe. Activation of SAPK/JNK signaling by protein kinase Cdelta in response to DNA damage. J Biol Chem 277:48372-8 (2002).

Yu, A., McMaster, C. R., Byers, D. M., Ridgway, N. D., and Cook, H. W. Stimulation of phosphatidylserine biosynthesis and facilitation of UV-induced apoptosis in Chinese hamster ovary cells overexpressing phospholipid scramblase 1. J Biol Chem, 278:9706-9714 (2003).

Yuan, Z. M., Utsugisawa, T., Ishiko, T., Nakada, S., Huang, Y., Kharbanda, S., Weichselbaum, R., and Kufe, D. Activation of protein kinase C delta by the c-Ab1 tyrosine kinase in response to ionizing radiation. Oncogene 16:1643-1648 (1998).

Zhao, J., Q. Zhou, T. Wiedmer, and P. J. Sims Level of expression of phospholipid scramblase regulates induced movement of phosphatidylserine to the cell surface. J Biol Chem 273:6603-6 (1998).

Zhou, G., M. L. Seibenhener, and M. W. Wooten. Nucleolin is a protein kinase C-zeta substrate. Connection between cell surface signaling and nucleus in PC12 cells. J Biol Chem 272:31130-7 (1997).

Zhou, Q., J. Zhao, J. G. Stout, R. A. Luhm, T. Wiedmer, and P. J. Sims Molecular cloning of human plasma membrane phospholipid scramblase. A protein mediating transbilayer movement of plasma membrane phospholipids. J Biol Chem 272:18240-4 (1997).

Zhou, Q., J. Zhao, T. Wiedmer, and P. J. Sims Normal hemostasis but defective hematopoietic response to growth factors in mice deficient in phospholipid scramblase 1. Blood 99:4030-8 (2002).

We claim:

1. A method of identifying a substance that upregulates production of PKC-δ in the presence of a proteasome inhibitor, comprising:
a) exposing cells expressing PKC-δ to the proteasome inhibitor;
b) exposing the cells of step a) to a test substance;
c) measuring the level of PKC-δ in the cells of step b); and
d) comparing the level of PKC-δ in the cells of step c) to a control level, wherein a higher level of PKC-δ indicates a substance that upregulates production of PKC-δ in the presence of the proteasome inhibitor.

2. The method of claim 1, wherein the proteasome inhibitor comprises bortezomib.

3. A composition comprising a proteasome inhibitor and a PKC-δ activator.

4. The composition of claim 2, wherein the proteasome inhibitor comprises bortezomib.

5. A composition comprising a proteasome inhibitor and a PKC-δ pathway activator.

6. The composition of claim 4, wherein the proteasome inhibitor comprises bortezomib.

7. A method of identifying a substance that increases localization of PKC-δ to mitochondria in the presence of bortezomib, comprising:
a) exposing cells expressing PKC-δ to bortezomib;
b) exposing the cells of step a) to a test substance;
c) measuring the level of PKC-δ in the cells of step b); and
d) comparing the level of PKC-δ in the cells of step c) to a control level, wherein a higher level of PKC-δ indicates a substance that increases localization of PKC-δ to mitochondria in the presence of bortezomib.

8. A method of identifying a substance that increases cleavage of PKC-δ in the presence of bortezomib, comprising:
a) exposing cells expressing PKC-δ to bortezomib;
b) exposing the cells of step a) to a test substance;
c) measuring the level of cleaved PKC-δ in the cells of step b); and
d) comparing the level of cleaved PKC-δ in the cells of step c) to a control level, wherein a higher level of cleaved PKC-δ indicates a substance that increases cleavage of PKC-δ in the presence of bortezomib.

9. A method of identifying a substance that blocks PKC-δ degradation in the presence of bortezomib, comprising:
a) exposing cells expressing PKC-δ to bortezomib;
b) exposing the cells of step a) to a test substance;
c) measuring the level of PKC-δ degradation in the cells of step b); and
d) comparing the level of degraded PKC-δ in the cells of step c) to a control level, wherein a lower level of degraded PKC-δ indicates a substance that blocks degradation of PKC-δ in the presence of bortezomib.

10. A method of identifying a substance that enhances tyrosine phosphorylation of PKC-δ in the presence of bortezomib, comprising:
a) exposing cells expressing PKC-δ to bortezomib;
b) exposing the cells of step a) to a test substance;
c) measuring the level of phosphorylated PKC-δ in the cells of step b); and
d) comparing the level of phosporylated PKC-δ in the cells of step c) to a control level, wherein a higher level of phosporylated PKC-δ indicates a substance that enhances tyrosine phosphorylation of PKC-δ in the presence of bortezomib.

11. A method of identifying a substance that blocks Src kinase, thereby activating PKC-δ, in the presence of bortezomib, comprising:
a) exposing cells expressing PKC-δ to bortezomib;
b) exposing the cells of step a) to a test substance;
c) measuring the level of Src kinase in the cells of step b); and
d) comparing the level of Src kinase in the cells of step c) to a control level, wherein a lower level of Src kinase indicates a substance that blocks Src kinase in the presence of bortezomib.

12. A method of treating a subject in need of such treatment, comprising administering to the subject an effective amount of bortezomib and a PKC-δ activator.

13. The method of claim 12, wherein the subject has cancer.

14. The method of claim 13, wherein the PKC-δ activator comprises phorbol ester.

15. The method of claim 13, wherein the PKC-δ activator comprises a mitochondria targeting agent.

16. The method of claim 12, wherein the PKC-δ activator comprises AD198.

17. A cell assay useful in identifying a substance that upregulates production of PKC-δ in the presence of bortezomib, wherein the cell expresses PKC-δ.

18. A method of treating a subject in need thereof, comprising administering to the subject a vector comprising PKC-δ, and administering to the subject an effective amount of bortezomib.

19. A method of detecting activation of PKC-δ comprising detecting phosphorylation of PLS3, wherein a higher level of PLS3 compared to a control indicates activation of PKC-δ.


SEQUENCE LISTING (3 sequences)

SEQ ID NO: 1 - protein, 676 aa, Artificial Sequence
  Description of Artificial Sequence/note = synthetic construct
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

SEQ ID NO: 2 - DNA, 2850 bp, Artificial Sequence
  Description of Artificial Sequence/note = synthetic construct
tcgcacttccgtgtgccgcggcgccggagcccgaggcggctgtagcccacatctcccgagcgacccccggcgcccgcccgccgcgcggaggcccgggccacacctcactggccgcttggcccatcccagtcagcgccgcgccgaaccccgtccgcgcgcgccggggagcggcgcccccgccgctgccgccgcgacccttggcgcctgcccctgcaacgggaggtctgcagggaactggccaggcaagggggcaggcccgtttctcctggtggttggtgcgttgtagcagcagcgggagccaggactaaggacaagcaggagctgggagccccagccccactgcaggccccaccatggcgccgttcctgcgcatcgccttcaactcctatgagctgggctccctgcaggccgaggacgaggcgaaccagcccttctgtgccgtgaagatgaaggaggcgctcagcacagagcgtgggaaaacactggtgcagaagaagccgaccatgtatcctgagtggaagtcgacgttcgatgcccacatctatgaggggcgcgtcatccagattgtgctaatgcgggcagcagaggagccagtgtctgaggtgaccgtgggtgtgtcggtgctggccgagcgctgcaagaagaacaatggcaaggctgagttctggctggacctgcagcctcaggccaaggtgttgatgtctgttcagtatttcctggaggacgtggattgcaaacagtctatgcgcagtgaggacgaggccaagttcccaacgatgaaccgccgcggagccatcaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacactatattccagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggccttgaaccaagtcacccagagagcctcccggagatcagactcagcctcctcagagcctgttgggatatatcagggtttcgagaagaagaccggagttgctggggaggacatgcaagacaacagtgggacctacggcaagatctgggagggcagcagcaagtgcaacatcaacaacttcatcttccacaaggtcctgggcaaaggcagcttcgggaaggtgctgcttggagagctgaagggcagaggagagtactttgccatcaaggccctcaagaaggatgtggtcctgatcgacgacgacgtggagtgcaccatggttgagaagcgggtgctgacacttgccgcagagaatccctttctcacccacctcatctgcaccttccagaccaaggaccacctgttctttgtgatggagttcctcaacgggggggacctgatgtaccacatccaggacaaaggccgctttgaactctaccgtgccacgttttatgccgctgagataatgtgtggactgcagtttctacacagcaagggcatcatttacagggacctcaaactggacaatgtgctgctggaccgggatggccacatcaagattgccgactttgggatgtgcaaagagaacatattcggggagagccgggccagcaccttctgcggcacccctgactatatcgcccctgagatcctacagggcctgaagtacacattctctgtggactggtggtctttcggggtccttctgtacgagatgctcattggccagtcccccttccatggtgatgatgaggatgaactcttcgagtccatccgtgtggacacgccacattatccccgctggatcaccaaggagtccaaggacatcctggagaagctctttgaaagggaaccaaccaagaggctgggagtgaccggaaacatcaaaatccaccccttcttcaagaccataaactggactctgctggaaaagcggaggttggagccacctttcaggcccaaagtgaagtcacccagagactacagtaactttgaccaggagttcctgaacgagaaggcgcgcctctcctacagcgacaagaacctcatcgactccatggaccagtctgcattcgctggcttctcctttgtgaaccccaaattcgagcacctcctggaagattgaggttcctggacagatcaggctagccctgccctccacccacacctgcccgctccccacgataagcaccagtgggactgtggtgacttctgctgctggccccgcccctgcccccagagcgtccttggctgccgtctggccgggctctcatggtacttcctctgtgaactgtgtgtgaatctgcttttcctctgccttcggagggaaattgtaaatcctgtgtttcattacttgaatgtagttatctattgaaaatatatattatatacatagacatatatatatatataataggctgtatatattgctcagtagagaaaaaccatgggggactggtgatatgttgatctttttcaaaaaaatatatatatgacaaaaaaaaaaaaaaaggagcacaagctgtttgaaccaccaggtttatttgtgtgtctaaataaacaccaaatagtaccaaaaaaaaaaaaaaaaa

SEQ ID NO: 3 - protein, 348 aa, Artificial Sequence
  Description of Artificial Sequence/note = synthetic construct
DNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED